(19) United States
(12) Patent Application Publication
(10) Pub. No.: US 2007/0105791 A1
(43) Pub. Date: May 10, 2007

(54) Method of treating clostridium difficile-associated diarrhea

(75) Inventors: Pamela Sears, San Diego, CA (US); Starr Louise Miller-Shangle, San Diego, CA (US); Robert Brian Walsh, Davis, CA (US); Youe-Kong Shue, Carlsbad, CA (US); Farah Babakhani, San Diego, CA (US); Thomas Louie, Calgary (CA); Chiu Yu-Hung, San Diego, CA (US); Alex Romero, San Diego, CA (US); Sherwood Gorbach, Weston, MA (US)

Correspondence Address: Kent H. Cheng, Suite 1210, 551 Fifth Avenue, New York, NY, 10176, US

(73) Assignee: Optimer Pharmaceuticals, Inc., San Diego, CA (US)

(21) Appl. No.: 11/585,521

(22) Filed: Oct. 23, 2006

Related U.S. Application Data
(63) Continuation-in-part of application No. 10/520,863, filed on Jul. 13, 2005.
(63) Continuation-in-part of application No. PCT/US05/16750, filed on May 13, 2005.
(63) Continuation-in-part of application No. PCT/US05/02887, filed on Jan. 31, 2005.
(60) Provisional application No. 60/729,135, filed on Oct. 21, 2005.
(60) Provisional application No. 60/749,641, filed on Dec. 12, 2005.
(60) Provisional application No. 60/399,956, filed on Jul. 29, 2002.
(60) Provisional application No. 60/570,697, filed on May 14, 2004.

(51) Int. Cl. A61K 31/70; A01N 43/04

(52) U.S. Cl. 514/35

(57) ABSTRACT

A method of treating a disease or disorder caused by the presence of a bacterium selected from the group consisting Clostridium species, Staphylococcus species, Enterococcus species and combinations thereof comprising administering to a patient in need an effective amount of a mixture, which comprises tiacumicin B, lipiarmycin A4, and at least one of other macrocyclic compounds:

### RELATED APPLICATIONS

This application claims benefit from U.S. Provisional Patent Application Ser. No. 60/729,135 which was filed on Oct. 21, 2005 and U.S. Provisional Application Ser. No. 60/749,641 which was filed Dec. 12, 2005. This application is continuation-in-part application of U.S. patent application Ser. No. 10/520,863 filed Jan. 11, 2005, which claims benefit of International Application No. PCT/US2003/021977 filed Jul. 15, 2003, which claims benefit of U.S. Provisional Application Ser. No. 60/399,956 filed Jul. 29, 2002. This application is also continuation-in-part application of International Application No. PCT/US05/16750 filed May 13, 2005, which claims benefit of U.S. Provisional Application Ser. No. 60/570,697 filed May 14, 2004. This application is yet continuation-in-part application of International Application No. PCT/US05/02887 filed Jan. 31, 2005.

The disclosures of the above-reference applications are incorporated by reference in their entirety herein.

### BACKGROUND OF THE INVENTION

1. Field of the Invention

This invention relates to the treatment of a disease caused by the presence of a bacterium selected from the group consisting of Clostridium species, Staphylococcus species, and Enterococcus species and combinations thereof, in particular a disease caused by the presence of a bacterium selected from the group consisting of Clostridium difficile (“C. difficile”), Clostridium perfringens (“C. perfringens”), Staphylococcus aureus (“S. aureus”) and combinations thereof, more particular a disease caused by the presence of C. difficile. The disease may be colitis, pseudomembranous colitis, or diarrhea.

2. Description of the Related Art

Antibiotic-associated diarrhea (AAD) is caused by toxin producing strains of C. difficile, S. aureus including methicillin-resistant Staphylococcus aureus (MRSA), and Clostridium perfringens (C. perfringens). AAD represents a major economic burden to the healthcare system that is conservatively estimated at $3-6 billion per year in excess hospital costs in the U.S. alone.

AAD is a significant problem in hospitals and long-term care facilities and in the community. C. difficile is the most common cause of AAD in the hospital setting, accounting for approximately 20% of cases of AAD and the majority of cases of antibiotic-associated colitis (AAC). The rising incidence of C. difficile associated diarrhea (CDAD) has been attributed to the frequent prescription of broad-spectrum antibiotics to hospitalized patients [Wilcox et al., Lancet 1996, 348: 767-8].

The most serious form of the disease is pseudomembranous colitis (PMC), which is manifested histologically by colitis with mucosal plaques, and clinically by severe diarrhea, abdominal cramps, and systemic toxicity. The overall mortality rate from CDAD is low, but is much greater in patients who develop severe colitis or systemic toxicity. A recent study has shown that even when death is not directly attributable to C. difficile, the rate of mortality in CDAD patients as compared to case-matched controls is much greater.

Diarrhea and colitis are caused by the elaboration of one or more C. difficile toxins. The organism proliferates in the colon in patients who have been given broad-spectrum antibiotics or, less commonly, cancer chemotherapy. CDAD is diagnosed in approximately 20% of hospitalized patients who develop diarrhea after treatment with such agents.

Current therapy for AAD or CDAD includes discontinuation of implicated antimicrobial or chemotherapy agents, nonspecific supportive measures, and treatment with antibiotics directed against C. difficile. The most common antimicrobial treatment options include vancomycin, and Metronidazole. Treatment of CDAD with antibiotics is associated with clinical relapse of the disease. Frequency of relapse is reported to be 5-50%, with a 20-30% recurrence rate being the most commonly quoted figure. Relapse occurs with nearly equal frequency regardless of the drug, dose, or duration of primary treatment with any of the antibiotics listed above. The major challenge in therapy is in the management of patients with multiple relapses, where antibiotic control is problematic.

The two most commonly utilized specific therapies are vancomycin and metronidazole, though vancomycin is the only drug approved by the FDA for this indication. However, Vancomycin is not recommended for first-line treatment of CDAD mainly because it is the only antibiotic active against some serious life-threatening multi-drug resistant bacteria. Therefore, in an effort to minimize the emergence of vancomycin-resistant Enterococcus (VRE) or vancomycin-resistant Staphylococcus aureus (VRSA), the medical community discourages the use of this drug except when absolutely necessary.

Metronidazole is recommended as initial therapy out of concern for the promotion and selection of vancomycin resistant gut flora, especially enterococci. Despite reports that the frequency of C. difficile resistance may be >6% in some countries, metronidazole remains nearly as effective as vancomycin, is considerably less expensive, and can be used either orally or intravenously. Metronidazole is associated with significant adverse effects including nausea, neuropathy, leukopenia, seizures, and a toxic reaction to alcohol. Furthermore, it is not safe for use in children or pregnant women.

Although both agents are effective in treating the infection, increasing rates of treatment failures and recurrence of diarrhea in approximately 20% of patients that initially respond are deficiencies of standard therapies. Therapy with metronidazole has been reported to be an important risk factor for VRE colonization and infection. In addition, the current treatment regime is rather cumbersome, requiring up to 500 mg qid for 10 to 14 days. Thus, there is a need for better treatment for cases of CDAD as well as for cases of other AAD and AAC.

Therefore, there is a need to develop a bactericidal drug with a low propensity to generate resistance, having reduced or no cross-resistance to existing antimicrobials and/or a prolonged post-antibiotic effect.

### SUMMARY OF THE INVENTION

The present invention provides a method of treating a disease or disorder caused by the presence of a bacterium selected from the group consisting of Clostridium species, Staphylococcus species, Enterococcus species and combinations thereof comprising administering to a patient in need an effective amount of a mixture. The mixture comprises an effective amount of tiacumicin B and an additional macrocycle selected from the group consisting of: 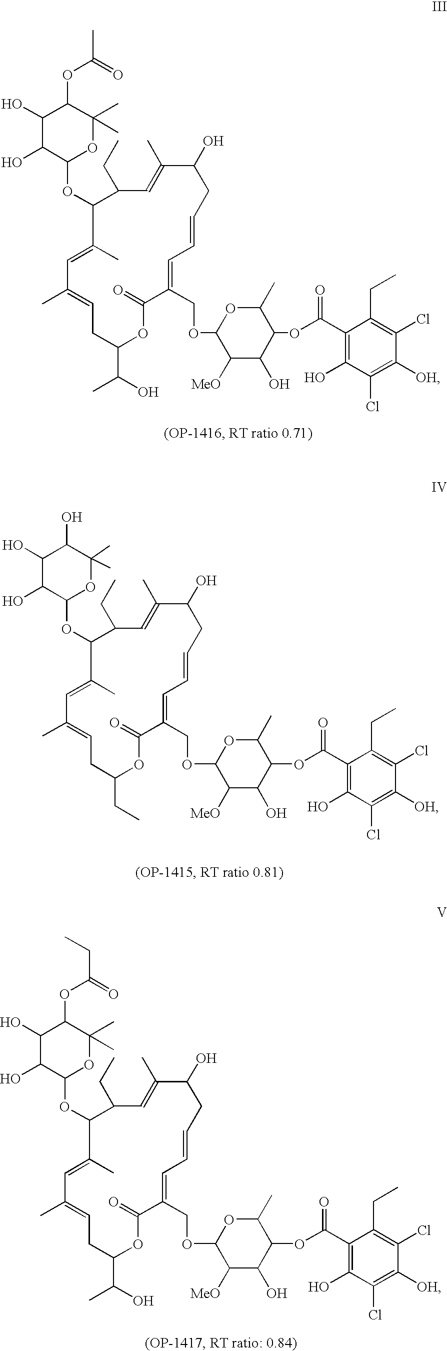 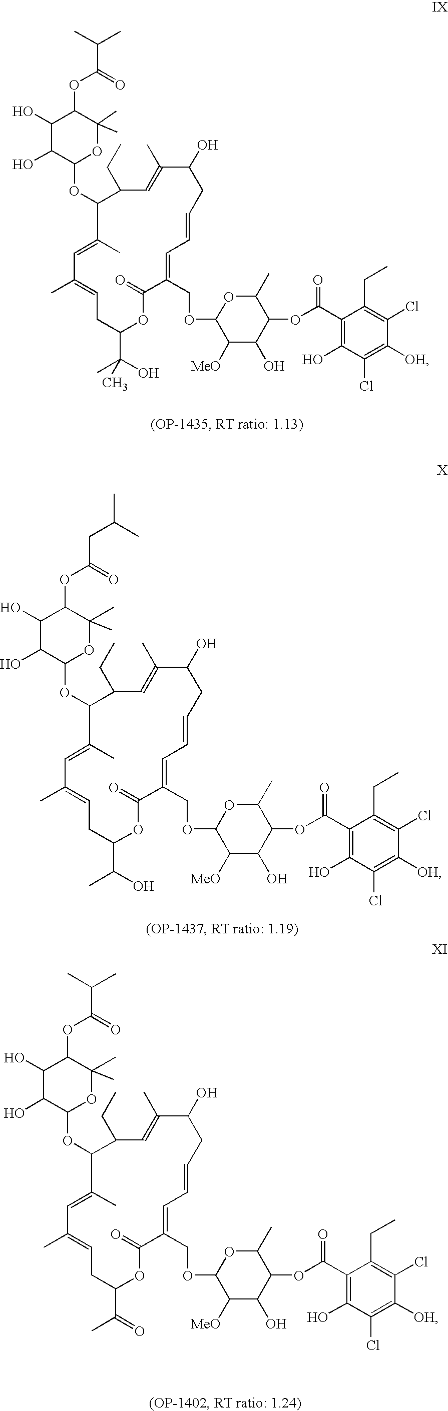 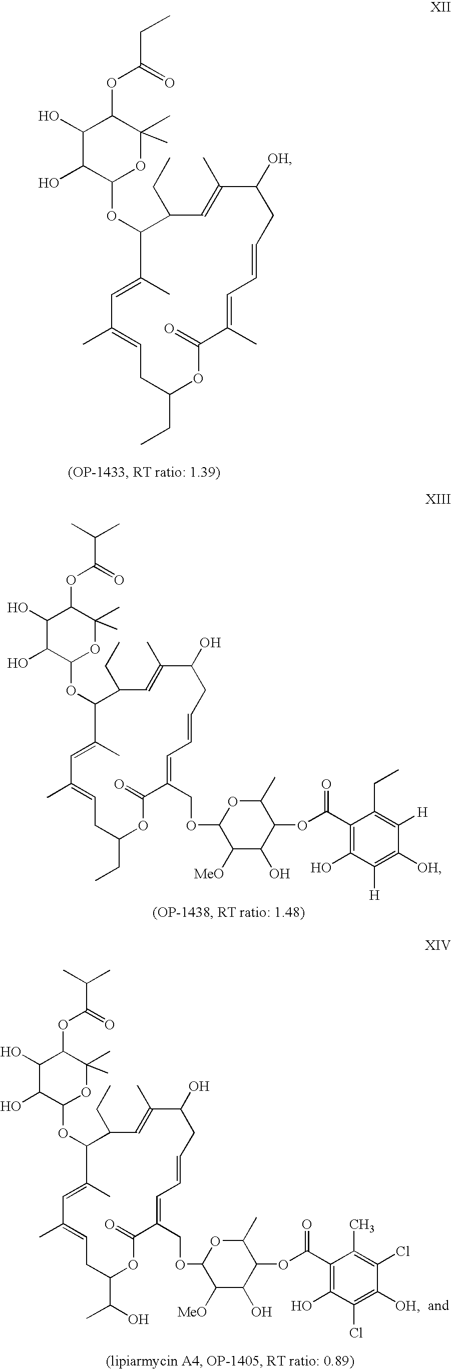
combinations thereof. When the compound of formula XIV is present, the mixture comprises about 0.1 to about 5% compound of formula XIV.

Preferably, the mixture comprises at least 90% tiacumicin B by weight. More preferably, the mixture comprises at least 95% tiacumicin B by weight.

Preferably, the mixture comprises at least 1%, more preferably, from about 2% to about 5%, of additional macrocycle by weight.

Preferably, the mixture comprises about 0.1% to about 5%, more preferably 0.3% to 3%, in particular 0.3% to 1.5%, especially about 1%, lipiarmycin A4 by weight.

Preferably, when liapiarmycin A4 is present, the mixture also comprises at least one of the following compounds:

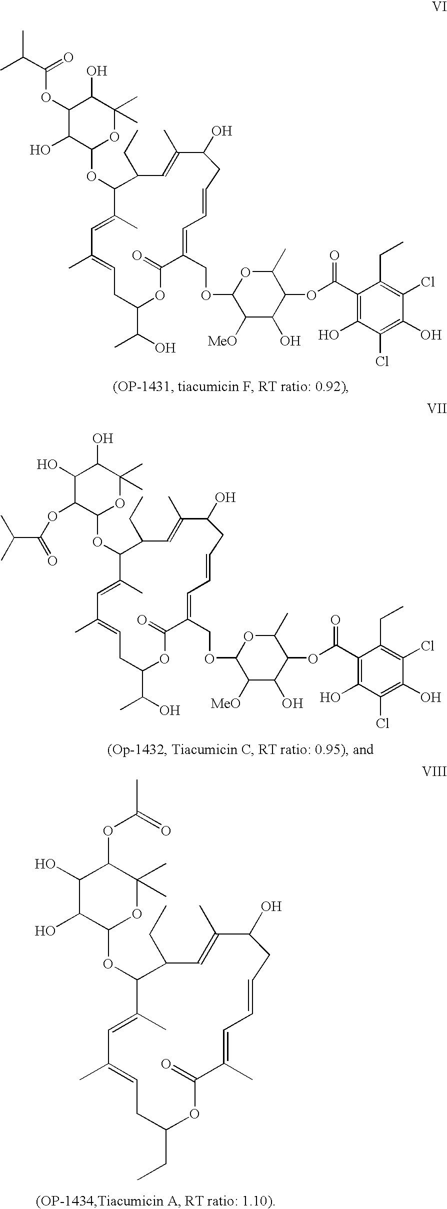

Preferably, the mixture exhibits an HPLC profile substantially depicted at FIG. 5

Preferably, the disease or disorder treated in accordance with the present invention is associated with C. difficile, C. perfringens, S. aureus, and combinations thereof. More preferably, the disease or disorder treated in accordance with the present invention is associated with C. difficile.

Preferably, the disease treated in accordance with the present invention is diarrhea or colitis, in particular diarrhea, more particularly CDAD.

Preferably, the mixture in accordance with the present invention is prepared by a process comprising:
culturing a microorganism in a nutrient medium to accumulate the mixture in the nutrient medium; and
isolating the mixture from the nutrient medium;
the nutrient medium comprises an adsorbent to adsorb the mixture.

The nutrient medium preferably comprises 0.5-15% of the adsorbent by weight. The absorbent is preferably an adsorbent resin. More preferably, the adsorbent resin is selected from the group consisting of Amberlite® XAD16, XAD16HP, XAD2, XAD7HP,XAD1180, XAD1600, IRC50, and Duolite® XAD761. The microorganism is preferably Dactylosporangium aurantiacum subspecies hamdenensis. The nutrient medium comprises, based on weight, 0.2% to 10% of glucose, 0.02% to 0.5% of K₂HPO₄, 0.02% to 0.5% of MgSO₄.7H₂O, 0.01% to 0.3% of KCl, 0.1% to 2% of CaCO₃, 0.05% to 2% of casamino acid, 0.05% to 2% of yeast extract, and 0.5% to 15% of XAD-16 resin. The culturing step is preferably conducted at a temperature from about 25 to about 35° C. and at a pH from about 6.0 to about 8.0.

Preferably, the disease treated in accordance with the present invention is associated with the use of antibiotics or cancer chemotherapies or antiviral therapy.

In accordance with one preferred embodiment, the mixture is administered in an amount of about 50 mg to 1000 mg, more preferably 100 mg to 400 mg, in particular 200 mg, one to three times daily, more preferably once or twice daily, in particular twice daily, within three to fifteen days, in particular around ten days. Oral administration is preferred.

The treatment of the present invention may allow for the effective treatment of diarrhea diseases associated with enterotoxigenic strains of C. difficile, S. aureus, and C. perfringens without compromising systemic antibiotics and without increasing vancomycin resistant enterococci (VRE) in the gut. The present invention also reduces the presence of VRE in the gut.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the Drawings:

FIG. 1 shows the Phase 1B-MD Dosing schedule.

FIG. 2 shows the bacteroides count following treatment. Pairs signed-ranks test, 2 tailed. For counts <3 log 10, a value of 2.9 was used.

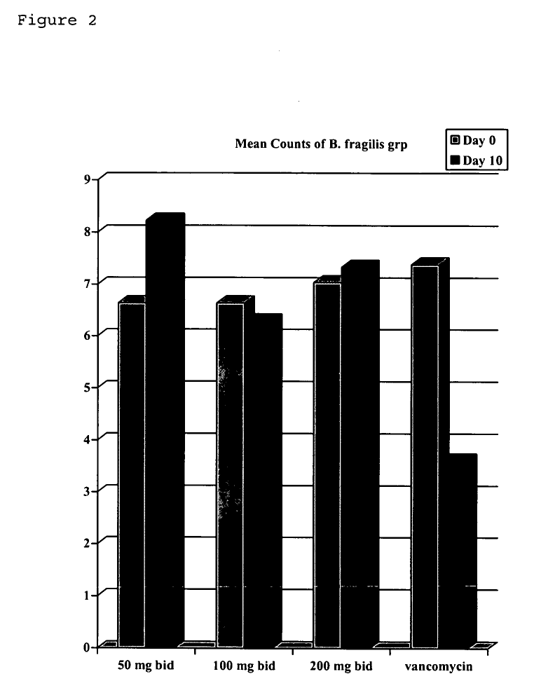

FIG. 3 shows the effect of Vancomycin therapy vs B. fragilis group.

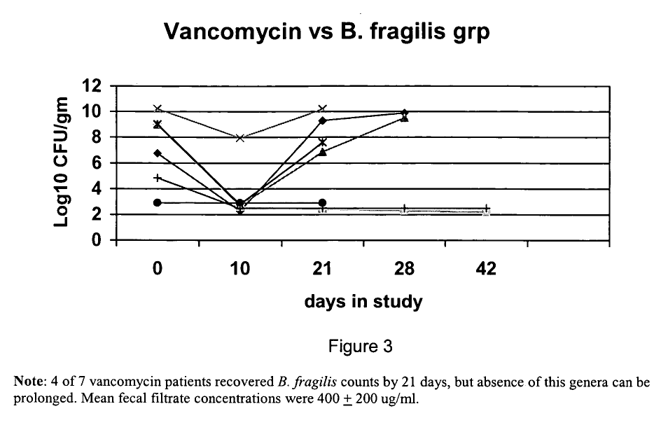

FIG. 4 shows the quantitative reduction of C. difficile vegetative counts after treatment with MCC.

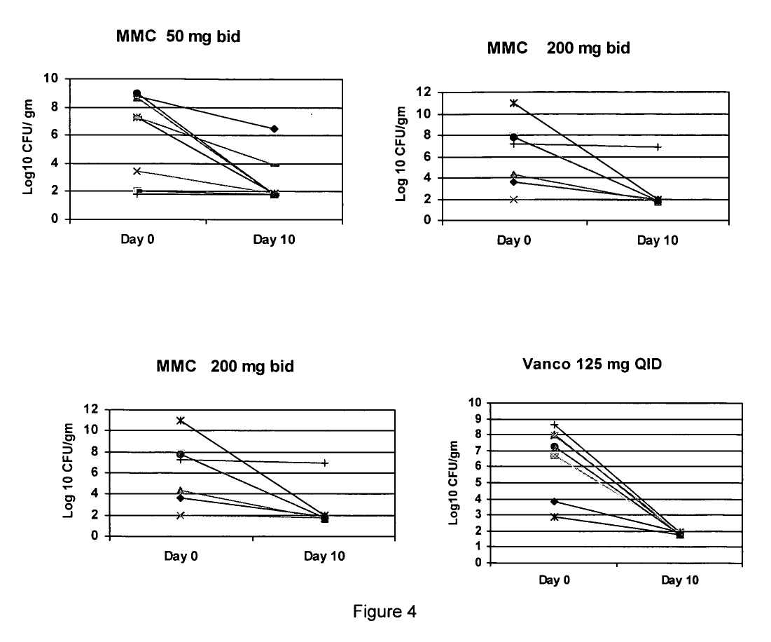

FIG. 5 is a typical HPLC profile of the mixture, which may be used in the method of the present invention. 7

### DETAILED DESCRIPTION OF THE PRESENTLY PREFERRED EMBODIMENTS

The definitions of certain abbreviations or terms used in the present application are provided as follows:

AAD=antibiotic-associated diarrhea

ATCC=American Type Culture Collection

¹³C=carbon 13

CO₂=carbon dioxide

N₂=nitrogen

H₂=hydrogen

TAPS=N-Tris(hydroxymethyl)methyl-3-aminopropanesulfonic acid

MOPS=3-(N-Morpholino)propanesulfonic acid

CDAD=Clostridium difficile-associated diarrhea

CLSI=Clinical and Laboratory Standards Institute, formerly NCCLS

ED₅₀=effective dose to produce 50% response

HPLC=high performance liquid chromatography

IR =infrared spectroscopy

LLOQ=lower limit of quantification

MCC=Macrocycle-Containing Composition

MIC=minimum inhibitory concentration

MIC₅₀=minimum inhibitory concentration to inhibit 50% of bacterial strains tested

MIC₉₀=minimum inhibitory concentration to inhibit 90% of bacterial strains tested

MRSA=methicillin-resistant Staphylococcus aureus

NCCLS=National Committee for Clinical Laboratory Standards, now CLSI

PMC=pseudomembranous colitis

VRE=vancomycin-resistant enterococci

VRSA=vancomycin-resistant Staphylococcus aureus

The term “antibiotic-associated condition” refers to a condition resulting when antibiotic therapy disturbs the balance of the microbial flora of the gut, allowing pathogenic organisms such as enterotoxin producing strains of C. difficile, S. aureus and C. perfringens to flourish. These organisms can cause diarrhea, pseudomembranous colitis, and colitis and are manifested by diarrhea, urgency, abdominal cramps, tenesmus, and fever among other symptoms. Diarrhea, when severe, causes dehydration and the medical complications associated with dehydration.

The term “MCC” refers to a preparation primarily containing tiacumicin B with respect to the whole antibiotic substance (e.g., at least 90%, preferably 95%-98% by HPLC assay). MCC also comprise a small amount (e.g., at least 1%, preferably 2%-5%) of tiacumicin B related compounds, i.e., lipiarmycin A4 and at least one of compound of formula III-XIV shown above. PCT application PCT/US03/21977, having an international publication number of WO 2004/014295 A2, provides a process of making a mixture comprising tiacumicin B. The entire content of this PCT application is incorporated herein as reference. However, MCC intended exclusively for use in non-humans may contain less than 80% of Tiacumicin B (with respect to the whole antibiotic substance, by HPLC assay).

The term “excipient” refers to an inert substance added to a pharmacological composition to further facilitate administration of a compound. Examples of excipients include but are not limited to, calcium carbonate, calcium phosphate, various sugars and types of starch, cellulose derivatives, gelatin, vegetable oils and polyethylene glycols.

The term “halogen” includes F, Cl, Br and I.

The term “macrocycles” refers to organic molecules with large ring structures usually containing over 10 atoms.

The term “18-membered macrocycles” refers to organic molecules with ring structures containing 18 atoms.

The term “membered ring” can embrace any cyclic structure, including carbocycles and heterocycles as described above. The term “membered” is meant to denote the number of skeletal atoms that constitute the ring. Thus, for example, pyridine, pyran and thiopyran are 6 membered rings and pyrrole, furan, and thiophene are 5 membered rings.

The term “MIC” or “minimum inhibitory concentration” refers to the lowest concentration of an antibiotic that is needed to inhibit growth of a bacterial isolate in vitro. A common method for determining the MIC of an antibiotic is to prepare several tubes containing serial dilutions of the antibiotic, that are then inoculated with the bacterial isolate of interest. Following incubation at appropriate atmosphere and temperature, the MIC of an antibiotic can be determined from the tube with the lowest concentration that shows no turbidity (no growth).

The term “MIC₅₀” refers to the lowest concentration of antibiotic required to inhibit the growth of 50% of the bacterial strains tested within a given bacterial species.

The term “MIC₉₀” refers to the lowest concentration of antibiotic required to inhibit the growth of 90% of the bacterial strains tested within a given bacterial species.

The term “patient” refers to a human or animal in need of medical treatment. For the purposes of this invention, human patients are typically institutionalized in a primary medical care facility such as a hospital or nursing home. However, treatment of a disease associated with the use of antibiotics or cancer chemotherapies or antiviral therapies can occur on an outpatient basis, upon discharge from a primary care facility, or can be prescribed by a physician for home-care, not in association with a primary medical care facility. Animals in need of medical treatment are typically in the care of a veterinarian.

The term “pharmaceutically acceptable carrier” refers to a carrier or diluent that is pharmaceutically acceptable.

The term “pharmaceutically acceptable salts” refers to those derived from pharmaceutically acceptable inorganic and organic bases. Salts derived from appropriate bases include alkali metal (e.g., sodium or potassium), alkaline earth metal (e.g., magnesium), ammonium and N(C₁-C₄ alkyl)₄⁺salts, and the like. Illustrative examples of some of these include sodium hydroxide, potassium hydroxide, choline hydroxide, sodium carbonate, and the like.

The term “pharmaceutical composition” refers to a mixture of one or more of the Tiacumicins described herein, or physiologically acceptable salts thereof, with other chemical components, such as physiologically acceptable carriers and/or excipients. The purpose of a pharmaceutical composition is to facilitate administration of a compound to an organism.

The term “physiologically acceptable carrier” refers to a carrier or diluent that does not cause significant irritation to an organism and does not abrogate the biological activity and properties of the administered compound.

The term “pseudomembranous colitis” or “enteritis” refers to the formation of pseudomembranous material (i.e., material composed of fibrin, mucous, necrotic epithelial cells and leukocytes) due to inflammation of the mucous membrane of both the small and large intestine.

The term “Tiacumicin” as used herein refers to a family of compounds all of which comprise the 18-membered macrocycle shown below in Formula I:

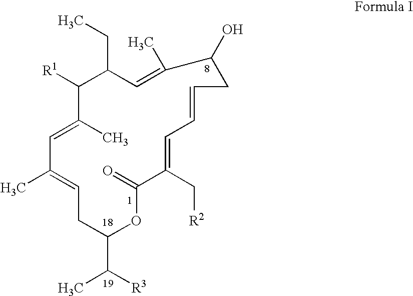

The term “Tiacumicin B” as used herein refers to the 18-membered macrocycle shown below in Formula II:

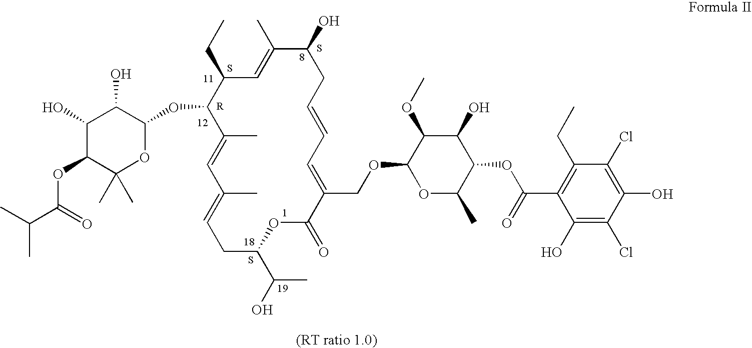

The term lipiarmycin A4 as used herein refers to the 1 8-membered macrocycle shown below in Formula XIV:

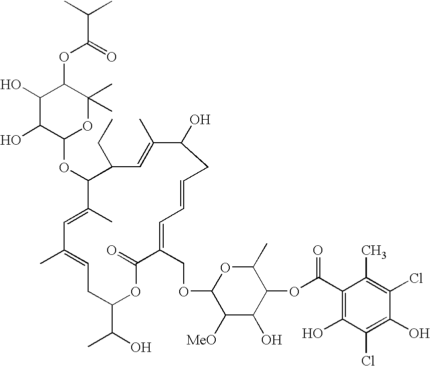

In accordance with one embodiment of the present invention, after multiple dose oral administrations, low MCC levels were detected in plasma, most of which fell below the limit of quantification. By contrast, fecal levels in both studies were extremely high, exceeding 10,000 times the MIC₉₀ (0.125 μg/mL) versus C. difficile.

In accordance with one embodiment of the present invention, recurrence of C. difficile-associated diarrhea can be inhibited in a patient by administering MCC in an amount and for a duration effective to inhibit recurrence of C. difficile but with a lack of effect on normal gut flora in the patient.

In accordance with one embodiment, the daily oral dosage of MCC for CDAD will range from between about 50 mg to about 1.0 grams of active agent per day, preferably, from between about 100 mg to about 600 milligrams per day. Generally, treatment will be continued for a time period ranging from between about 3 to about 15 days. Greater or lesser amounts of drug and treatment intervals may be utilized as required. For example, according to the results of a clinical study hereinafter reported, a dosage of about 100-400 milligrams of MCC per day, over the course of from about ten days, proved effective in treating CDAD with minimal clinical recurrence.

In accordance with one embodiment of the present invention, the mixture can be made by the following general process.

MCC-producing bacteria was grown in vessels ranging from shake flasks to large “batch” fermenters. For producing substantial quantities of MCC, submerged aerobic fermentation in tanks is utilized. However, small amounts may be obtained by shake-flask culture. For tank fermentation, it is preferable to use a vegetative inoculum. The vegetative inoculum is prepared by inoculating a small volume of culture medium with the spore form, mycelial fragments, or a lyophilized pellet of the organism to obtain a fresh, actively growing culture of the organism. The vegetative inoculum is then transferred to a larger tank where, after a suitable incubation time, the MCC antibiotic is produced in much improved yield. It may be necessary to add small amounts of an antifoam agent to large-scale fermentation media if foaming becomes a problem.

The production proceeds in a control medium with other additives/ingredients to improve the production. A liquid-submerged, stirred-culture process is used for the production of MCC. Fermentation is carried out at a temperature range of 25° C. to 37 ° C. The consumption of the carbon source is carefully monitored and an additional amount of carbon source is added as needed. The pH of the fermentation is preferably maintained between about 6.0 to about 8.0. MCC is produced and accumulated between 3 to 15 days after inoculation of the fermentation.

Commercially available adsorbent resins were found to enhance the yield and recovery efficiency of MCC during the fermentation. Adsorbents are preferably present in the range between 0.5-15% by weight. MCC was recovered in exceptional yield (>100 mg/L broth) from the fermentation broth by resin absorption and eluted from the resin and mycelium by washing with solvents of various polarities

MCC was first captured from the broth during fermentation using adsorbent resins such as Amberlite resin (XAD-16). Upon completion of fermentation, the solid mass (including the adsorbent resin) is separated from the broth by sieving. The solid mass are eluted with ethyl acetate then concentrated under reduced pressure.

Upon completion of fermentation, the solid mass (including the adsorbent resin) is separated from the broth by sieving. MCC is eluted from the resin with organic solvents such as ethyl acetate, methanol, acetonitrile or a mixture of two or more organic solvents. The extract is then concentrated under reduced pressure. This residue is further purified by trituration with low polarity solvents such as hexanes, heptanes, methylcyclohexane, or by partitioning between two phase solvent systems such as: ethyl acetate/water; ethyl acetate/aqueous sodium chloride solution; methanol/hexane, acetonitrile/hexane or other mixtures of two or more solvents in various ratios and combinations or by column chromatography eluting with an appropriate organic solvent system. The current purification process of MCC is based on medium-pressure reverse-phase (C-18) column using 50:50:1 CH₃CN/H₂O/AcOH or 70:30:1, MeOH/H₂O/AcOH as eluent. The fractions contain desired MCC were washed with brine and the concentrated. The residue was dissolved in ethyl acetate and washed with water and organic layer was evaporated to dryness to provide a pale yellow foam which was again washed with isopropyl alcohol and dried under reduced pressure to yield a white powder. Combine fractions having purity >88%. Concentrate fractions to one-half of original volume. Filter precipitate and wash filter cake with water. The solid was dried under high vacuum overnight to give a white powder and analyzed by HPLC. Typically, the mixture comprising tiacumicin B as major components ranged from 90% to 99%, lipiarmycin A4 (0.1% to 5%), and at least one or more of the macrocycles of formula III-XIV described above.

### EXAMPLES

The following examples are provided by way of describing specific embodiments of the present invention without intending to limit the scope of the invention in any way.

The mixture used in the following examples is prepared in accordance with the process of making described above. The following table shows composition of several exemplary mixtures made in accordance with the present invention.

API mixture profile

| | strain | | | | | | | | | | |
|---|---|---|---|---|---|---|---|---|---|---|---|
| | wild type | wild type | wild type | wild type | wild type | wild type | wild type | mutant | mutant | | |
| | Trial | | | | | | | | | | |
| | | | | | | | | n/a | | | |
| | 1A | 1B | 1B | 1B | 2A | 2A | 2A | (crude) | 2B | | fold increased |
| | Lot # | | | | | | | | | | in MIC |
| | 92161001 | 92161901 | 92161902 | 92161903 | 93161001 | 93161901 | 93161002 | F7502 | B-0660041 | | relative to |
| RRT | % | % | % | % | % | % | % | % | % | Compound | tiacumicn B |
| 0.32 | — | 0.06 | 0.07 | — | — | 0.06 | — | — | 0.06 | | |
| 0.39 | — | — | 0.07 | — | — | — | — | — | 0.02 | | |
| 0.49 | — | 0.19 | 0.13 | — | — | — | 0.15 | — | 0.05 | | |
| 0.71 | — | — | — | — | — | — | — | 3.23 | 0.02 | OP-1416 | 2-4x |
| 0.75 | 0.48 | 0.28 | 0.16 | 0.17 | 0.32 | 0.08 | 0.12 | 0.49 | 0.35 | | |
| 0.79 | 0.11 | 0.09 | 0.07 | 0.06 | 0.05 | 0.08 | — | 0.72 | 0.21 | | |
| 0.81 | 0.08 | — | — | — | — | — | — | 1.01 | 0.08 | OP-1415 | 8-16x |
| 0.84 | 0.05 | 0.04 | 0.18 | 0.12 | — | 0.87 | — | 3.96 | 0.25 | OP-1417 | 2-4x |
| 0.86 | — | — | — | — | — | — | — | — | — | | |
| 0.88 | — | — | — | — | — | — | — | 3.20 | — | | |
| 0.89 | 0.24 | 0.37 | 0.61 | 0.32 | 0.69 | | 0.50 | 4.85 | 1.13 | OP-1405 | 1-4x |
| | | | | | | | | | | (lipiarmycin A4) | |
| 0.92 | 0.44 | 0.51 | 0.42 | 0.21 | 0.19 | 0.16 | 0.17 | 1.74 | 0.15 | OP-1431 | 8x |
| | | | | | | | | | | (tiacumicin F) | |
| 0.95 | — | — | — | — | — | — | 0.08 | 0.63 | 0.06 | OP-1432 | 8-16x |
| | | | | | | | | | | (tiacumicin C) | |
| 0.96 | 0.27 | 0.44 | 0.65 | 0.35 | — | — | 0.15 | — | — | | |
| 0.98 | 0.25 | — | — | — | — | — | — | — | — | | |
| 1.00 | 95.54 | 97.26 | 96.40 | 98.78 | 98.16 | 98.55 | 98.36 | 73.61 | 95.6 | OP-1441 | 1x |
| | | | | | | | | | | (tiacumicin B) | |
| 1.03 | — | — | — | — | — | — | 0.22 | — | 0.22 | | |
| 1.04 | 0.29 | — | — | — | — | — | — | — | — | | |
| 1.05 | 0.37 | 0.24 | 0.60 | — | — | — | — | — | — | | |
| 1.07 | 0.43 | — | 0.16 | — | 0.08 | — | — | — | 0.09 | | |
| 1.10 | 0.90 | 0.31 | 0.36 | — | 0.26 | 0.10 | 0.10 | 0.37 | — | OP-1434 | >32x |
| | | | | | | | | | | (tiacumicin A) | |
| 1.11 | — | — | — | — | — | — | — | — | 0.81 | | |
| 1.13 | — | — | — | — | — | — | — | — | 0.55 | | |
| 1.13 | 0.32 | 0.20 | 0.13 | — | 0.24 | 0.11 | 0.14 | 1.64 | — | OP-1435 | 2x |
| 1.14 | 0.19 | — | — | — | — | — | — | — | — | | |
| 1.19 | 0.04 | — | — | — | — | — | — | 1.50 | — | OP-1437 | 2x |
| 1.23 | — | — | — | — | — | — | — | 2.61 | 0.30 | OP-1402 | 2-4x |

The HPLC assay is conducted in accordance with the following procedure.

Mobile Phase A: Add 2.0 mL of trifluoroacetic acid to 2L of HPLC water, filter and degas.

Mobile Phase B: Add 1.0 mL of trifluoroacetic acid to 2L of Acetonitrile, filter and degas.

Column: 4.6×150 mm column that contains octyl silane chemically bonded to porous silica or ceramic micro-particles 3 to 10 μm in diameter (e.g., Zorbax Eclipse XDB-C8, 3.5 μm).

Detector: 230 nm.

Flow rate: About 1.0 mL/min.

Injection volume: About 10 μL.

Run time: About 10 μL.

Diluent: 100% acetonitrile.

| | | % | |
|---|---|---|---|
| | Time (Min) | Mobile Phase A | Mobile Phase B |
| Gradient program: | 0 | 60 | 40 |
| | 3.0 | 50 | 50 |
| | 14.0 | 39 | 61 |
| | 14.5 | 60 | 40 |

Note:
Retention time of the mixture must be within 8-12 minutes.

Standard Preparation: Accurately weigh about 20 mg of the mixture into a 100 mL volumetric flask, dissolve in and dilute to volume with Diluent.

Sample Preparation: Accurately weight about 20 mg of the mixture in a 100 mL volumetric flask. Add about 60 mL Diluent and vortex to dissolve. Dilute to volume with Diluent and mix.

System Suitability: Chromatography the Standard preparation and record the peak responses as directed under Procedure. The relative standard deviation of tiacumicin B peak areas for five replicate injections is NMT 2.0%, the tailing factor of tiacumicin B areas is NMT 2.0.

Procedure: Inject about 10 μL of Diluent. Separately inject equal volumes (about 10 μL) of Standard and Sample preparations, record the chromatograms and measure the detector responses for major peaks.

Relative Retention Time:

| Related Substance | RT ratio |
|---|---|
| Compound of formula II (tiacumicin B) | 1.0 |
| Compound of formula III | 0.71 |
| Compound of formula IV | 0.81 |
| Compound of formula V | 0.84 |
| Compound of formula VI (tiacumicin F) | 0.92 |
| Compound of formula VII (Tiacumicin C) | 0.95 |
| Compound of formula VIII (Tiacumicin A) | 1.10 |
| Compound of formula IX | 1.13 |
| Compound of formula X | 1.19 |
| Compound of formula XI | 1.24 |
| Compound of formula XII | 1.39 |
| Compound of formula XIII | 1.48 |
| Compound of formula XIV (Lipiarmycin A4) | 0.89 |

Calculations: Calculate the assay value using the following formula:

$$Assay , \% = \frac{{R}_{u}}{{R}_{s}} \times \frac{{W}_{std} ( mg )}{StdDil ( mL )} \times P \times \frac{SmpDil ( mL )}{{W}_{smp} ( mg ) \times WF} \times 100$$

Where:
Rᵤ=tiacumicin B peak area obtained from the assay preparation.
Rₛ=tiacumicin B peak area obtained from the Standard preparation.
P=Purity of Reference standard, including water factor.
W_{std}=Standard weight (mg)
StdDil=Standard dilution (mL).
Wₛₘₚ=Sample weight (mg).
WF=Sample water factor.

Discard peaks originated fro Diluent and calculate the percentage w/w of individual and total related substances by the formulae:

$$Individual related substance ( \% w \text{/} w ) = \frac{{R}_{i}}{{R}_{u}} \times {RF}_{i} \times 100$$

Where:
Rᵢ=Related substances peak area obtained from the Sample Preparation.
Ru=tiacumicin B peak area obtained from the Sample Preparation.
RFᵢ=Related Substance response factor (RFᵢ=1.0 for all related substances.)

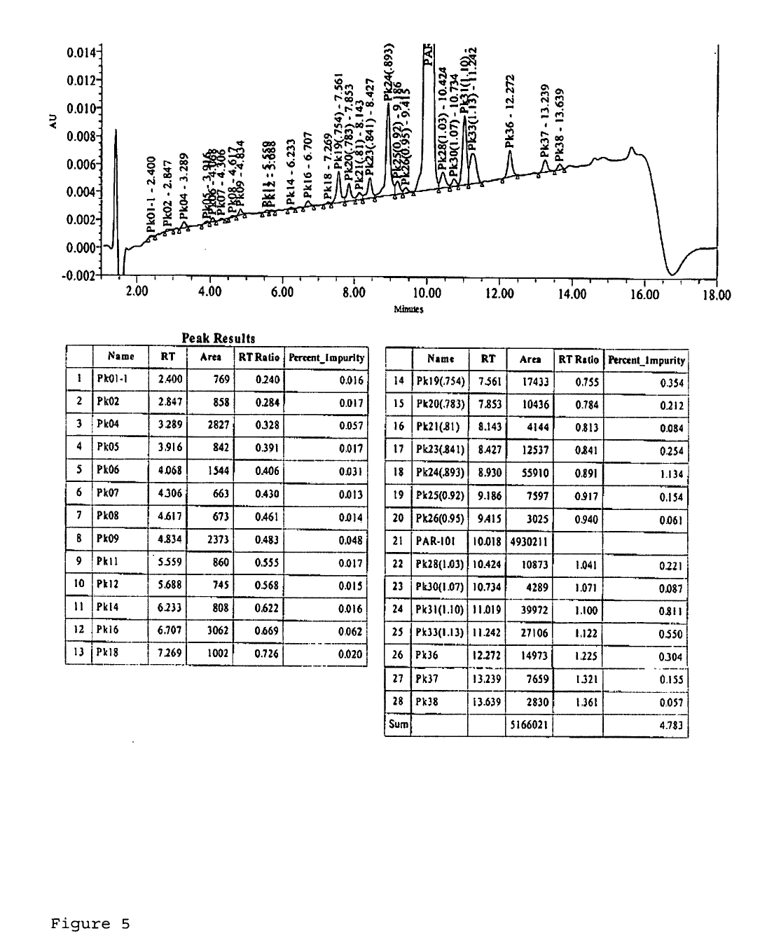

In addition, a typical HPLC profile of the mixture in accordance with the present invention is shown in FIG. 5. The compounds contained in the mixture, e.g., compounds of formula II-XIV, may be found in the HPLC profile based on their RT ratio. Par-101 in FIG. 5 represents tiacumicin B with RT ratio being 1.0.

The above mixture (50 mg) is then mixed with 100 mg Avicel PH 102, FMC (microcrystalline cellulose) in a size 1 capsule shell.

### Example 1

### Effect of Inoculum, pH, and Cations on the In Vitro Activity of MCC Vs Clostridium difficile

The MIC values measured for many antibiotics are known to be affected by environmental variables such as pH, the concentration of divalent cations such as calcium and magnesium, and the bacterial density. The dependence of the antibacterial activity on these factors is an important consideration, particularly for an antibiotic that targets bacteria in the gut, where these parameters can vary greatly with the diet and disease state. m

The sensitivity of the MIC to these environmental variables may also be an important factor to consider when designing methodology for future in vitro testing. The Clinical and Laboratory Standards Institute, CLSI (formerly NCCLS) recommends using Brucella agar supplemented with vitamin K₁ and hemin for Minimal Inhibitory Concentration (MIC) determination for anaerobes. The level of divalent cations in this medium, however, is not standardized. Moreover, the pH of the media used under anaerobic glove box may also vary under different gas mixtures. Anaerobes are typically incubated in a mixture of nitrogen, hydrogen, and carbon dioxide, and the presence of CO₂ will acidify the medium and can be a significant source of variability. The inoculum size may also be difficult to standardize given the variety of atmospheric conditions available for anaerobic susceptibility testing (H₂/CO₂ generator, evacuation/replacement method, or anaerobic chamber). The anaerobic conditions available to each lab will determine the duration of organism exposure to aerobic atmosphere during bench top manipulations and anaerobic equilibration, and thus affect culture viability and experimental result.

In this study, we examined the effect on MIC of the level of the divalent cations calcium and magnesium, pH (from 5-8), inoculum density (over 3 orders of magnitude), and also the variability from lot to lot of Brucella broth .

Materials and Methods

Bacterial Strains:

Laboratory strains of Clostridium difficile 9689, 700057, 43255, 17857 and Eubacterium lentum 43055 were obtained from American Type Culture Collection (ATCC). All strains were streaked onto brucella agar plates, supplemented with hemin, and vitamin K from frozen stocks maintained at − at 78° C. in 10% glycerol prior to use.

MIC Testing:

Current CLSI procedures (4) for anaerobic broth and agar dilution were used for MIC evaluation. Broth dilution is not a validated method for MIC testing of Clostridium; however, due to potential inaccuracy of measuring the pH of solid agar after equilibration inside the anaerobic chamber, both methods were used and compared for the assessment of pH effects.

Inoculum Density Effect on MIC Values:

The effects of inoculum density on susceptibility of C. difficile to MCC and vancomycin were determined using the agar dilution method (4). The inocula were prepared by first making a suspension of ˜10⁸ cfu/mL and then serially diluting the suspension by 10-fold factors to obtain a culture density range between 10⁵-10⁸ cfu/mL, to give spot densities of 10²-10⁵ cfu/spot.

pH Effect on MIC Values:

The susceptibility of C. difficile to MCC was evaluated over a pH range of 6-8 using both agar dilution and microbroth dilution methods.

Using the agar dilution method, the MIC of MCC was determined over a pH range of 6.2-8.0 against C. difficile strains in two separate experiments. In order to achieve the desired anaerobic pH for susceptibility testing, buffer (100 mM of NaH₂PO₄ or TAPS [N-Tris(hydroxymethyl)methyl-3-aminopropanesulfonic Acid]) was added to media at pH 7 and 8, respectively. Even with strong buffering, the pH shifted slightly following equilibration in the anaerobic gas, and thus in some cases media was titered in ambient air to above the desired anaerobic pH. The actual pH was always confirmed following equilibration inside the anaerobic chamber. Vancomycin, used as a control, was tested only at pH 7.

Using the broth microdilution method, the MIC values of MCC and vancomycin were determined over a pH range of 6-8 against C. difficile strains in 3 separate series. In the first series, unbuffered Brucella broth was titrated in ambient air to obtain a pH range from 5-9. However, anaerobic equilibration of media in the glove box environment (10% H₂/5% CO₂/85% N₂) lowered the pH of the media, resulting in an anaerobic pH range from 5-7.5 (as tested using a portable pH meter with a flat-bottomed pH probe calibrated with buffer standards outside the glove box, then transferred inside). For subsequent experiments, buffer was added to media to resist pH shifts caused by anaerobic equilibration. In the second series, 10 mM buffer [NaH₂PO₄ .H₂O pH 7.0, MOPS pH 8.0, or TAPS pH 9.0, pH values in ambient air) was added to media with pH values greater than 6 to obtain a pH range from 6-7.6 after anaerobic equilibration. In the third series, the buffer concentration was increased to 100 mM for pH treatments above 6 to obtain an anaerobic pH range from 6-8.1.

Divalent Cation Concentration Effect on MIC Values:

The agar dilution method was used to determine the effect of calcium and magnesium ion concentrations on susceptibility of C. difficile strains to MCC. The level of divalent cations in the Brucella broth as acquired from the manufacturer were determined by the Laboratory Specialists, Inc. Additional amounts of divalent cations were added (in the form of calcium or magnesium chloride) in order to give calcium ion concentrations of 2.1, 3.0 and 5.7 mg/dL and magnesium ion concentrations of 3.3, 4.5, and 7.5 mg/dL.

Reproducibility of MCC MIC Values with Different Commercial Lots of Media:

Using the CLSI agar dilution method, susceptibility of C. difficile to MCC was also examined with three different commercial lots of Brucella agar, from BBL (lot #30768960, 211086, and 3167036), supplemented with different lots of vitamin K (Sigma lot #V-3501 and 0214010) and hemin (Sigma lot #072K1221 and 034K7656).

Results

Inoculum Density Effect on MIC Values:

Tables 1 and 2 demonstrate the effect of inoculum density on the MIC of MCC and vancomycin against two strains of C. difficile (ATCC 9689 and ATCC 700057). Susceptibility of both C. difficile strains to MCC was unaffected by inoculum concentration from 10⁵-10⁸ cfu/ml (102-10⁵ CFU/spot), as shown by identical MIC values obtained for all inoculum concentrations tested. The MIC of vancomycin, however, increased progressively with increasing inoculum concentration, with the highest inoculum density showing a fourfold increase in MIC over the lowest inoculum density. These results demonstrate that inoculum density is not a significant factor affecting the outcome of MCC susceptibility testing of C. difficile.

TABLE 1
In vitro activity of MCC (μg/mL) vs. different inoculum densities
of C. difficile ATCC 9689 (10²-10⁵ CFU/spot).

| | |
|---|---|
| Inoculum Density | ATCC 9689 |

| (cfu/ml) | CFU/spot | MCC | vanc |
|---|---|---|---|
| 1.92 × 10⁸ | 1.92 × 10⁵ | 0.063 | 2 |
| 1.92 × 10⁷ | 1.92 × 10⁴ | 0.063 | 1 |
| 1.92 × 10⁶ | 1.92 × 10³ | 0.063 | 1 |
| 1.92 × 10⁵ | 1.92 × 10² | 0.063 | 0.5 |

TABLE 2
In vitro activity of MCC (μg/mL) vs. different inoculum densities
of C. difficile ATCC 700057 (10²-10⁵ CFU/spot).

| | | |
|---|---|---|
| Inoculum Density | Inoculum Density | ATCC 700057 |

| (cfu/ml) | (cfu/ml) | MCC | vanc |
|---|---|---|---|
| 1.48 × 10⁸ | 1.48 × 10⁵ | 0.125 | 1, 2 |
| 1.48 × 10⁷ | 1.48 × 10⁴ | 0.125 | 1 |
| 1.48 × 10⁶ | 1.48 × 10³ | 0.125 | 1 |
| 1.48 × 10⁵ | 1.48 × 10² | 0.125 | 0.5 |

pH Effect on MIC Values:

Table 3 depicts the effect of various pH values on susceptibility of C. difficile to MCC as measured by agar dilution method on two separate days. During the first run, the highest pH treatment (pH 7.9) showed an 8-fold increase in MIC values over the lower pH treatments (pH 6.2 & pH 7.2) for both strains of C. difficile. When a confirmatory run was repeated at the highest pH (pH 8.0), the MIC value remained high for both strains. No increase in MCC MIC was observed between pH 6.2 and pH 7 for either strain.

The increase in MIC values with pH did not consistently correlate with increased growth, thus the effect of pH on MIC did not appear to be merely due to the enhanced viability of the organism at higher pH. The pH 7 treatment had less dense organism spot growth relative to the pH 6.2 and pH 7.9 treatments.

TABLE 3
pH effects on agar dilution MIC values (buffered medium)

| | | Anaerobic pH | | | |
|---|---|---|---|---|---|
| | | | 7 | 7.9 | 8.0 |
| | | 6.2 | 100 mM NaH₂PO₄ | 100 mM TAPS | 100 mM TAPS |
| Organism | Drug | Unbuffered | pH 7.2 (Air) | pH 9.2 (Air) | pH 9.2 (Air) |
| ATCC 9689 | MCC | 0.063 | 0.063 | 0.5 | 1 |
| | Vancomycin | 1 (pH 6.7) | 4 | | |
| ATCC 700057 | MCC | 0.125 | 0.125 | 1 | 2 |
| | Vancomycin | 2 (pH 6.7) | 4 | | |

Table 4, 5 and 6 represents MIC data from the broth microdilution susceptibility method performed on three separate days with pH ranges from 5 to 8.1. In the first series, in which the medium was unbuffered, the MIC of MCC at pH 7.5 was 8×greater than the MIC at pH 5.9 for both C. difficile strains (Table 4). The MIC at pH 5 could not be determined, because the organism failed to grow at this pH. The buffered (10 mM) pH 7.6 treatment showed 8-fold and 16-fold increases in MCC MIC over the pH 6 treatment for C. difficile ATCC 9689 & ATCC 700057, respectively (Table 5). In the third, strongly buffered (100 mM) series, similar results were seen with the highest pH treatment (pH 8.1) showing a 16-fold increase in MIC over the lowest pH treatment (pH 6) for both organisms (Table 6). Vancomycin showed a similar trend with the highest pH treatment producing MICs 4-8 fold greater than the lowest pH treatment in all three experiments.

TABLE 4
pH effects on MIC using unbuffered media

| | | Anaerobic pH (unbuffered) | | | | |
|---|---|---|---|---|---|---|
| Organism | Drug | 5 | 5.9 | 6.6 | 7.1 | 7.5 |
| ATCC 9689 | MCC | no growth | ≦0.016 | ≦0.016 | 0.063 | 0.125 |
| | MCC | | ≦0.016 | 0.031 | 0.063 | 0.125 |
| | vanc | | 0.5, 1 | 0.5, 1 | 2 | 4 |
| | vanc | | 1 | 0.5, 1 | 2 | 4 |
| ATCC 700057 | MCC | no growth | 0.031 | 0.063 | 0.125 | 0.25 |
| | MCC | | 0.031 | 0.063 | 0.125 | 0.25 |
| | vanc | | 0.5 | 1 | 1 | 2, 4 |
| | vanc | | 0.5 | 1 | 1, 2 | 2, 4 |

TABLE 5
pH effects on MIC using weakly buffered media (10 mM)

| | | Anaerobic pH | | | |
|---|---|---|---|---|---|
| | | | 6.7 | 7.2 | 7.6 |
| | | 6 | 10 mM NaH₂PO₄ | 10 mM MOPS | 10 mM TAPS |
| Organism | Drug | pH 6.0 (Air) | pH 7.0 (Air) | pH 8.0 (Air) | pH 9.0 (Air) |
| ATCC 9689 | MCC | ≦0.016 | 0.031 | 0.063 | 0.125 |
| | MCC | ≦0.016 | 0.031 | 0.063 | 0.125 |
| | vanc | 0.5 | 1 | 2 | 4 |
| | vanc | 0.5 | 1 | 2 | 4 |
| ATCC 700057 | MCC | 0.031 | 0.063 | 0.125 | 0.5 |
| | MCC | 0.031 | 0.063 | 0.25 | 0.5 |
| | vanc | 0.5 | 1 | 2 | 4 |
| | vanc | 0.5 | 1 | 2 | 4 |

TABLE 6
pH effects on MIC using strongly buffered media (100 mM)

| | | Anaerobic pH | | | | |
|---|---|---|---|---|---|---|
| | | | 6.8 | 7.5 | 8 | 8.1 |
| | | 6 | 100 mM NaH₂PO₄ | 100 mM MOPS | 100 mM TAPS | 100 mM TAPS |
| Organism | Drug | pH 6.0 (Air) | pH 7.0 (Air) | pH 8.0 (Air) | pH 9.0 (Air) | pH 9.5 (Air) |
| ATCC 9689 | MCC | ≦0.016, 0.031 | 0.031 | 0.125 | 0.25, 0.5 | 0.25, 0.5 |
| | MCC | ≦0.016 | 0.031 | 0.125 | 0.25 | 0.25 |
| | vanc | 1 | 1 | 4 | >8 | >8 |
| | vanc | 0.5 | 1, 2 | 4 | >8 | >8 |
| ATCC 700057 | MCC | 0.031, 0.063 | 0.063, 0.125 | 0.25 | 1 | 0.5 |
| | MCC | 0.031 | 0.063, 0.125 | 0.25 | 0.5, 1 | 0.5 |
| | vanc | 1 | 2 | 4 | 8 | 8 |
| | vanc | 1 | 2 | 4 | 8 | 8 |

Assay plates at all pH treatments were also visually examined for overall growth. In the first series, which utilized unbuffered broth, overall culture turbidity increased with increasing pH. The same trend was observed in the second series, which utilized 10 mM buffered broth, except the culture turbidity was the same for pH 7.2 and pH 7.6. In the third series, culture turbidity was more equivalent across the pH treatments, with the exception of pH 7.5, which was the most turbid.

Overall, with both methods of susceptibility testing and across varying concentrations of buffer salts, the MIC values of MCC and vancomycin increased with increasing pH for both strains of C. difficile.

Divalent Cation Concentration Effect on MIC Values:

Measurement of the calcium and magnesium levels in commercial Brucella broth showed calcium and magnesium ion concentration of 21 and 33 mg/L, respectively. Various additional amounts of divalent cations were added, and MCC MIC values for C. difficile strains were tested at three different concentrations of calcium ions (21, 30 and 57 mg/L) and three different concentrations of magnesium ions (33, 45 and 75 mg/L). The MIC values remained the same in all types of media. C. difficile 9689 had MIC value of 0.063 μg/ml and C. difficile 700057 with MIC value of 0.125 μg/ml in media with varying concentrations of cations. Vancomycin, which was tested as a control with supplemented Brucella agar without any extra calcium or magnesium as control during the experiments, demonstrated the expected MIC value of 1 μg/ml for all runs (Tables 7 and 8).

TABLE 7
In vitro activity of MCC in supplemented Brucella agar with
different divalent cation concentrations

| | Calcium concent in | C. difficile | C. difficile |
|---|---|---|---|
| | Brucella agar | (ATCC 700057) | (ATCC 9689) |
| Drug | media (mg/L) | MIC (μg/mL) | MIC (μg/mL) |
| MCC | 33 | 0.125 | 0.063 |
| | 45 | 0.125 | 0.063 |
| | 75 | 0.125 | 0.063 |
| Vancomycin | 33 | 1 | 1 |

TABLE 8
In vitro activity of MCC in supplemented Brucella agar with different
divalent cation concentrations

| | Magnesium concent | C. difficile | C. difficile |
|---|---|---|---|
| | in Brucella agar | (ATCC 700057) | (ATCC 9689) |
| Drug | media (mg/L) | MIC (μg/mL) | MIC (μg/mL) |
| MCC | 21 | 0.125 | 0.063 |
| | 30 | 0.125 | 0.063 |
| | 57 | 0.125 | 0.063 |
| Vancomycin | 21 | 1 | 1 |

MCC MIC Values with Different Commercial Lots of Media:

Three different lots of supplemented Brucella agar media were used on three separate days to compare the activity of MCC against C. difficile strains. The MIC assays were controlled by testing the activity of the QC organism, Eubacterium lentum vs. clindamycin which was within the CLSI (NCCLS) acceptable ranges, i.e. 0.06-0.25 μg/mL. Another control step for the MIC assays was to include metronidazole and monitor its activity vs. C. difficile strains, which in our laboratory has been shown to have MIC values ranging between 0.25-0.5 μg/mL. As shown in Table 9, the activity of MCC vs. C. difficile was not affected by different lots of supplemeted Brucellaagar . All controls demonstrated activities within established ranges.

TABLE 9
In vitro activity of MCC tested with three different lots of media

| | MIC values (μg/mL) | | | | | | | | |
|---|---|---|---|---|---|---|---|---|---|
| Bacteria | Metronidazole | | | Clindamycin | | | MCC | | |
| (ATCC #) | Day 1 | Day 2 | Day 3 | Day 1 | Day 2 | Day 3 | Day 1 | Day 2 | Day 3 |
| C. difficile (9689) | 0.5 | 0.5 | 0.5 | 4 | 4 | 4 | 0.25 | 0.25 | 0.125 |
| C. difficile (43255) | 0.5 | 0.5 | 0.5 | 8 | 4, 8 | 8 | 0.25 | 0.5 | 0.25 |
| C. difficile (17857) | 0.25 | 0.5 | 0.5 | 4 | 2 | 4 | 0.125, 0.25 | 0.25 | 0.125 |
| Eubacterium | 1 | 0.25 | 1 | 0.25 | 0.25 | 0.125 | 0.125, 0.25 | 0.25 | 0.06, 0.125 |
| lentum (43055) | | | | | | | | | |

Conclusions

In contrast to vancomycin, the activity of MCC vs. C. difficile was unaffected by inoculum concentrations, in the range of 10²-10⁵ cfu/spot.

The susceptibility of C. difficile to MCC was unaffected by cation concentrations (calcium ion in the range of 2.1-5.7 mg/dL and magnesium concentration of 3.3-7.5 mg/dL), and by various commercial lots of media.

The MIC values for both MCC and vancomycin increased with increasing pH over a pH range of 6-8. The high MIC values at basic pH may be due to deprotonated form of the phenolic hydroxyl groups of both compounds above their pKa, where they form a charged species that is expected to be less permeable to bacterial cells. In contrast, below the pKa (7.22 for MCC), the antibiotics will be mostly protonated, and thus should permeate the cell membrane more efficiently.

Organism density generally increased with increasing pH; the dependence of growth density, but not MIC, on pH was reduced in the presence of buffering agents. Though organism density was positively correlated with basicity in the absence of buffer, it is unlikely that MIC trends are the result of the effect of pH on organism density alone. This is because the same relationship between MIC and pH was observed in buffered experiments where organism density was more equivalent across pH treatments, presumably due to the differential effect of buffer type on organism growth.

### Example 2

### Safety, Pharmacokinetics and Outcomes of MCC in Healthy Subjects and Patients with Clostridium difficile-Associated Diarrhea (CDAD)

Phase 1B-MD.

Synopsis. This was an oral, multiple-dose, double-blind, randomized, placebo-controlled, dose escalation study conducted at the University of Miami Division of Clinical Pharmacology, Miami, Fla. Richard Preston, M.D. served as the Principal Investigator for this trial. The tolerability and pharmacokinetics of multiple oral doses of MCC were evaluated in a total of 24 healthy volunteer subjects. The oral doses of MCC evaluated (in 3 groups of 8 subjects each, with 6 active and 2 placebo) were 150, 300, and 450 mg (in powder-filled capsules containing 50 mg of study drug) administered daily after a morning breakfast for 10 consecutive days. Subjects were dosed and monitored on a combined inpatient/outpatient basis. Subjects were admitted to the research unit on Day 0 and again on Day 9 of the 10-day dosing period, and stayed for up to 48 hours after each admission. Subjects were discharged on Day 2 and Day 11 after completing the scheduled events and procedures. During the outpatient period, subjects reported daily to the research unit for dosing and stayed for 3 hours under observation.

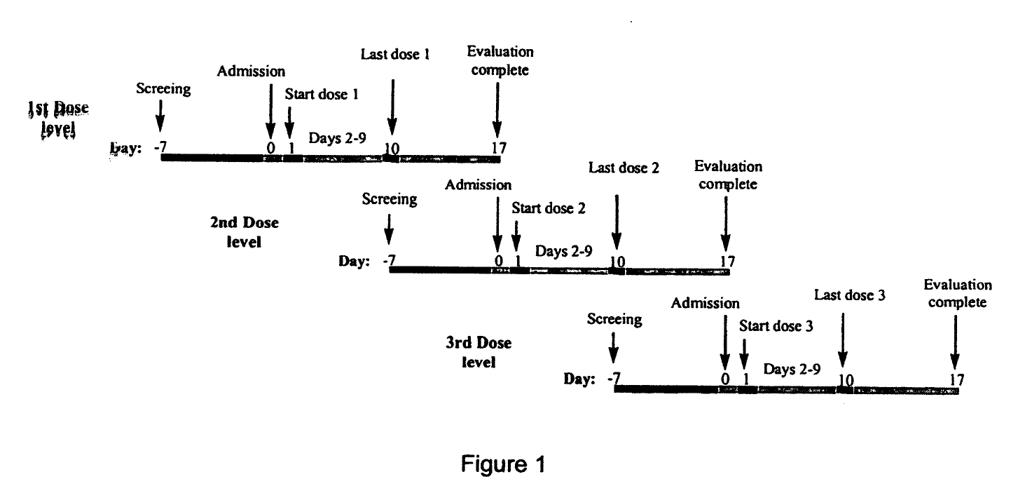

Serial blood, urine, and fecal samples were collected at various time points/intervals during the multiple dosing periods. Plasma, urine, and fecal concentrations of MCC were determined for pharmacokinetic analysis. A follow-up examination was scheduled on Day 17 of each study period before subjects exited from the study. Study subjects were closely monitored for the occurrence of any adverse experiences or abnormal laboratory test findings throughout the treatment periods and at the study follow-up. See, FIG. 1, Phase 1B-MD Dosing schedule.

Phase 2A.

Synopsis. This was a dose-finding study to select a safe and effective dose of MCC. Subjects were randomized to receive either 100 (50 mg every 12 hours), 200 (100 mg every 12 hours), or 400 (200 mg every 12 hours) mg/day for 10 days followed by clinical evaluation. Subjects recorded all symptoms on daily diary cards. Particular attention was to be given to stool frequency and consistency, the presence of blood in the stool, and abdominal discomfort. Laboratory assessments were performed at Screening for entry and at End of Treatment (Day 10-12) or withdrawal (whichever was sooner). Clinical observation and diary card evaluation were performed at End of Treatment (Day 10-12). Patient interviews were conducted on treatment Days 2 through 9, Day 17, and Day 52. For entry inclusion criteria, assay for Clostridium difficile toxin was performed. For subjects that failed to respond to MCC treatment, and in the event of clinical recurrence, both C. difficile toxin assay and culture were performed. Clinical, laboratory, and microbiological assessments were also performed at exit for subjects that failed to respond to treatment. Pharmacokinetic plasma samples were taken 0.5 hr prior to dosing and 2 hr after dosing on the first and last days of dosing.

Key Inclusion Criteria. Subjects were patients with C.difficile associated diarrhea as defined by: 1) diarrhea (a change in bowel habits, with 3 or more unformed bowel movements in 24 hours, or more than 6 loose or watery stools within 36 hours.) and 2) presence of either toxin A or B of C. difficile in the stool.

Key Exclusion Criteria Subjects could not have 1) severe or life-threatening CDAD 2) life-threatening or serious disease unrelated to CDAD, 3) concurrent use of: vancomycin, metronidazole, bacitracin, or related drugs. (If the Investigator felt the clinical imperative to begin treatment before knowing the laboratory result for stool toxin, up to 24 hours, but no more than 3 doses, of treatment with metronidazole and/or vancomycin was to be allowed.); any drugs used for the treatment of CDAD; or other antibiotics 4) history of ulcerative colitis or Crohn's disease and multiple recurrences (defined as more than one recurrence) of CDAD within the past three months. (Subjects with a single recurrence of CDAD were permitted to enroll.)

Schedule of Events

TABLE 10
Schedule of Evaluation Procedures in the phase 2A study

| | Day 1 | Treatment | End of | | |
|---|---|---|---|---|---|
| | Screen/ | Days 2 | Treatment | Completion | Day 52 |
| Assessments | Enrollment | through 9 | or Day 10 | Day 17 | Follow-up |
| Informed Consent | X | | | | |
| Inclusion/Exclusion | X | | | | |
| Medical History | X | | | | |
| Physical Examination | X | | X | | |
| Vital Signs | X | | X | | |
| 12-Lead ECG | X | | X | | |
| Clinical Laboratory Tests | X | | X | | |
| Stool Sample | X | | X | | |
| PK Blood Sampling^{a} | X | | X | | |
| Fecal PK Sampling | | | X | | |
| Adverse Events | | X | X | X | |
| Concomitant Medication | X | X | X | X | X |
| Pregnancy Test | X | | | | |
| Diary Card Review | X | X | X | | |
| Study Medication | | | | | |
| Administration | X | X | X | | |
| Subject Interview | X | X | X | X | X |

^{a}Blood samples for pharmacokinetics taken 0.5 hr prior to and 2 hr post administration on the first and last days of dosing

Endpoints. At the end of therapy, the investigator determined if the subject had been cured of failed. In addition, the time to resolution of diarrhea (defined as resolution to <3 loose or watery stools per day) and the complete relief of symptoms of CDAD by day 10 of therapy (complete relief was resolution to ≦3 total stools per day, whether loose or firm; and absence of fever, elevated white blood cells, or abdominal pain) were tracked as primary endpoints, and recurrence within 6 weeks following therapy (recurrence of diarrhea, defined as 3 or more loose/watery stools per day, with a positive toxin test) was tracked as a secondary endpoint.

Analysis

Safety Population:

The safety population was to include all randomized subjects who received at least one dose of study medication and had safety information available.

Efficacy Population:

Clinical success or failure was determined in patients treated per protocol. The population analyzed for time to resolution of diarrhea and complete relief of symptoms was the modified intent to treat population (mITT), consisting of all randomized subjects who received at least one dose of study medication, had a history of diarrhea, and had 3 or more loose stools in 24 hours and a positive C. difficile toxin at baseline.

Time to resolution of diarrhea was defined as time (in days) from the first dose of study medication to the resolution of diarrhea; time to resolution of diarrhea was compared among the three treatment groups. The cessation day of diarrhea was defined as the first day that <3 unformed stools (watery or loose) within a 24 hour period occurred and was sustained for the duration of treatment up to study Day 10. Resolution of diarrhea was assessed during a 10 to 12 day period utilizing the subject diary data.

Complete Relief of Symptoms of CDAD:

Complete relief of symptoms of CDAD was defined as resolution to <3 bowel movements per day (as recorded on the patient diary) without other associated signs/symptoms such as fever (≧37.7° C.), abdominal pain (no response on diary) and elevated WBC (normal laboratory range of WBC) by Day 10 of the study. If any variable was missing, this outcome was considered unknown.

Clinical Recurrence Rate:

Clinical recurrence was defined as >3 unformed stools (loose or watery) and a positive stool for C. difficile toxin A or B within 6 weeks posttreatment.

Results

Enrollment and Demographics

The following sections summarize the enrollment and demographic characteristics of the study populations in the phase 1B-MD and 2A trials. A total of 24 healthy subjects were enrolled for the phase 1B-MD study. Alternate male and female subjects were enrolled to provide an even split between the sexes. Subjects ranged in age from 38-62 years (average 51.6±7.5 yr), in weight from 55.5-90 kg (average 71.5±9.2 kg), and in height 147-183 cm (average 164.8±10.8 cm.)

In the phase 2B study, a total of 49 subjects were enrolled. One subject withdrew consent and was dropped from the study prior to receiving any study drug, and was not evaluable for either safety or efficacy. One subject (400 mg dosing group) had >6 bowel movements in 36 hours, but <3 bowel movements in the prior 24 hours, and could not be evaluated for time to resolution of diarrhea but was evaluable for clinical response and safety analyses. Three patients were discontinued after 1 or 2 doses due to removal of consent (1 subject, 100 mg dosing group), requirement for additional antibiotics for pneumonia (1 subject, 100 mg dosing group), or inability to take study medication (1 subject, 200 mg dosing group). Subject demographics are listed in Table 11.

TABLE 11
Summary demographics for the Phase 2A study; demographics for the 48
subjects in the population evaluable for safety are shown.

| | MCC | MCC | MCC | |
|---|---|---|---|---|
| | 100 mg/Day | 200 mg/Day | 400 mg/Day | All Subjects |
| | (N = 16) | (N = 16) | (N = 16) | (N = 48) |
| Sex | | | | |
| Female | 10 (62.5%) | 11 (68.8%) | 9 (56.3%) | 30 (62.5%) |
| Male | 6 (37.5%) | 5 (31.3%) | 7 (43.8%) | 18 (37.5%) |
| Race | | | | |
| Caucasian | 14 (87.5%) | 15 (93.8%) | 14 (87.5%) | 43 (89.6%) |
| Black | 1 (6.3%) | 1 (6.3%) | 0 (0.0%) | 2 (4.2%) |
| Asian | 0 (0.0%) | 0 (0.0%) | 1 (6.3%) | 1 (2.1%) |
| Hispanic | 0 (0.0%) | 0 (0.0%) | 0 (0.0%) | 0 (0.0%) |
| Other^{a} | 1 (6.3%) | 0 (0.0%) | 1 (6.3%) | 2 (4.2%) |
| Age(Yrs) | | | | |
| Mean ± SD | 56.3 ± 17.78 | 53.1 ± 22.97 | 55.3 ± 17.69 | 54.9 ± 19.26 |
| Median | 54.5 | 55.5 | 56.0 | 56.0 |
| Range | 28.0-89.0 | 18.0-88.0 | 18.0-90.0 | 18.0-90.0 |
| Weight(Kg) | | | | |
| Mean ± SD | 69.2 ± 14.00 | 68.4 ± 11.46 | 67.5 ± 13.56 | 68.4 ± 12.82 |
| Median | 69.3 | 66.0 | 65.2 | 66.0 |
| Range | 38.0-89.0 | 52.0-96.0 | 40.0-88.2 | 38.0-96.0 |
| Height(cm) | | | | |
| Mean ± SD | 163.8 ± 15.52 | 166.4 ± 9.48 | 166.2 ± 13.18 | 165.5 ± 12.80 |
| Median | 162.1 | 170.0 | 163.8 | 165.0 |
| Range | 122.0-187.5 | 150.0-178.0 | 142.0-193.0 | 122.0-193.0 |
| Calculated Body Mass Index^{b} | | | | |
| Mean ± SD | 25.8 ± 3.89 | 24.9 ± 4.50 | 24.3 ± 2.52 | 25.0 ± 3.68 |
| Median | 25.0 | 24.0 | 24.5 | 25.0 |
| Range | 17.0-34.0 | 17.0-32.0 | 20.0-28.0 | 17.0-34.0 |

NOTE:
values represent number of subjects unless otherwise indicated.
^{a}Other includes: East Indian, Indian.
^{b}Calculated body mass index is defined as (weight in kg)/(height in meters)².

Efficacy

In the clinical evaluation of treatment success or failure at the end of therapy, two patients in the low dosing group (2/14), 2 patients in the mid dosing group (2/15), and no patients in the top dosing group (0/16) were considered treatment failures by the investigator. Among the subjects (n=41) that were treatment successes, CDAD recurred in one subject (1/12) in the 100 mg/day dosing group and one subject (1/16) in the top dosing group, for a recurrence rate of 2/41 (5%) overall. Both recurrences occurred approximately 1 month following the end of therapy.

TABLE 12
Rates of clinical cure and recurrence in
the population treated per protocol.

| | MCC | | MCC | | MCC | |
|---|---|---|---|---|---|---|
| | 100 mg/Day | | 200 mg/Day | | 400 mg/Day | |
| | N | % | N | % | N | % |
| Total | 14 | 100 | 15 | 100 | 16 | 100 |
| Treatment success | 12 | 86 | 13 | 87 | 16 | 100 |
| Treatment failure | 2 | 14 | 2 | 13 | 0 | 0 |
| Clinical recurrence^{a} | 1 | 8.3 | 0 | 0 | 1 | 6.3 |

^{a}Recurrence of toxin-posivite diarrhea within 6 weeks post-treatment, evaluated in patients that were clinical successes.

The time to resolution of diarrhea was defined as the time for the patient to resolve to less than 3 unformed stools per day, according to the patient's diary card. In the mITT population, the median time to relief was 5.5 days, 3.5 days, and 3.0 days for the MCC 100 mg/day, 200 mg/day and 400 mg/day treatment groups, respectively. The mean time to resolution of diarrhea in days was 6.3±3.66 days in 100 mg/day-treated subjects, 4.8±3.56 days in 200 mg/day-treated subjects, and 3.6±2.03 in 400 mg/day-treated subjects. There was no statistically significant difference in time to resolution of diarrhea between the 100 mg/day and 200 mg/day treatment groups, and between the 200 mg/day and 400 mg/day treatment groups; however the difference between the 100 mg/day and 400 mg/day treatment groups approached statistical significance (p=0.0506 Kaplan Meier estimate and p=0.0503 Kruskal-Wallis test).

TABLE 13
Time to Resolution of Diarrhea (mITT population), defined as time to
resolve to <3 unformed bowel movements per day (according to the
patient's diary card)

| | MCC | MCC | MCC | |
|---|---|---|---|---|
| | 100 mg/Day | 200 mg/Day | 400 mg/Day | P-Value |
| N | 16 | 16 | 15 | |
| N (Resolved Diarrhea) | 10 | 12 | 14 | |
| N (Censored: Did not resolve)^{a} | 4 | 3 | 1 | |
| N (Censored: Dropped from study) | 2 | 1 | 0 | |
| N (Censored: Total) | 6 | 4 | 1 | |
| Median Time (Days)^{b} | 5.5 | 3.5 | 3.0 | |
| P-Value^{c} | | | | 0.1912 |
| MCC 100-MCC 200^{c} | | | | 0.2901 |
| MCC 100-MCC 400^{c} | | | | 0.0506 |
| MCC 200-MCC 400^{c} | | | | 0.6143 |

^{a}Subjects whose diarrhea was not resolved to <3 loose stools/day by day 10
^{b}Kaplan-Meier estimates
^{c}P-value obtained from generalized Wilcoxon Test.

Complete relief of symptoms of CDAD by the end of treatment, defined as ≦3 total bowel movements per day (whether formed or unformed, as recorded on the patient's diary card), and no fever, elevated WBC count, or abdominal pain (according to response on patient diary card) by the 10^{th} day of the study, is shown in Table 14. Complete relief was achieved by 37.5% of the 100 mg/day treatment group, 50.0% of the 400 mg/day treatment group, and 86.7% of the 400 mg/day treatment group. It is worth noting that most patients that did not have complete relief by day 10 were nevertheless treatment successes, had resolution of symptoms by day 17, and did not require further treatment. Three patients that dropped from the study (one for removal of consent, one for the requirement of exclusionary antibiotics, and one for the inability to take oral medications) are also listed as having no complete relief.

TABLE 14
Complete Relief of Symptoms of CDAD by end of therapy in the
mITT population, defined as resolution to ≦3 total bowel movements
per day (formed or unformed, as noted on the patient's diary card)
without other associated signs/symptoms such as fever, abdominal
pain, and elevated WBC by Day 10 of the study

| | MCC | | MCC | | MCC | |
|---|---|---|---|---|---|---|
| | 100 mg/Day | | 200 mg/Day | | 400 mg/Day | |
| | n | % | n | % | n | % |
| Complete Relief | 6 | (37.5) | 8 | (50.0) | 13 | (86.7) |
| No Complete Relief | 9 | (56.3) | 6 | (37.5) | 2 | (13.3) |
| Required further treatment | 2 | (12.5) | 2 | (12.5) | 0 | (0) |
| Required no further | 5 | (31.3) | 3 | (18.8) | 2 | (13.3) |
| treatment | | | | | | |
| Dropped from study | 2 | (12.5) | 1 | (6.3) | 0 | (0) |
| Unknown | 1 | (6.3) | 2 | (12.5) | 0 | (0.0) |

Only 2 subjects (1 subject in the 100 mg/day treatment group and 1 subject in the 400 mg/day treatment group) experienced clinical recurrence.

Safety

In the phase 1B-MD study, MCC was well tolerated by all subjects at all doses. Fourteen adverse events were reported, 7 in the 150 mg group, 2 in the 450 mg group, and 5 in the placebo group. The adverse events are summarized as follows: headache (2), dizziness (1), weakness (1), fatigue (1), nasal congestion (1), difficulty swallowing (1), pharyngitis (1), conjunctivitis (1), upper respiratory infection (2), rash (1), and pruritis (1). No subjects receiving MCC had adverse events considered to be drug-related.

In the phase 2A study, as shown in Table 15, 4/16 (25.0%) subjects in the 100 mg/day treatment group, 4/16 (25.0%) subjects in the 200 mg/day treatment group, and 1/16 (6.3%) subjects in the 400 mg/day treatment group, reported at least one AE during the study. The highest frequency of AEs was reported in the infections and infestations body system in the 100 mg/day treatment group (3/16; 18.8% subjects). There were 2/16 (12.5%) subjects who reported vascular disorders in the 100 mg/day treatment group and 2/16 (12.5%) subjects who reported gastrointestinal disorders in the 200 mg/day treatment group.

TABLE 15
Incidence of adverse events in the safety population of the 2A
study, summarized by system organ class and preferred term

| | MCC | | MCC | | MCC | |
|---|---|---|---|---|---|---|
| | 100 mg/Day | | 200 mg/Day | | 400 mg/Day | |
| System Organ Class | (N = 16) | | (N = 16) | | (N = 16) | |
| Preferred Term | n | (%) | n | (%) | n | (%) |
| Total subjects with adverse events | 4 | (25.0) | 4 | (25.0) | 1 | (6.3) |
| Cardiac disorders | 1 | (6.3) | 0 | (0.0) | 0 | (0.0) |
| Cardiac failure congestive | 1 | (6.3) | 0 | (0.0) | 0 | (0.0) |
| Gastrointestinal disorders | 0 | (0.0) | 2 | (12.5) | 0 | (0.0) |
| Gastrointestinal haemorrhage | 0 | (0.0) | 1 | (6.3) | 0 | (0.0) |
| Pancreatitis chronic | 0 | (0.0) | 1 | (6.3) | 0 | (0.0) |
| General disorders and administration site conditions | 1 | (6.3) | 1 | (6.3) | 0 | (0.0) |
| Chest pain | 1 | (6.3) | 1 | (6.3) | 0 | (0.0) |
| Infections and infestations | 3 | (18.8) | 1 | (6.3) | 0 | (0.0) |
| Bronchitis | 1 | (6.3) | 0 | (0.0) | 0 | (0.0) |
| Infection | 1 | (6.3) | 0 | (0.0) | 0 | (0.0) |
| Pneumonia | 1 | (6.3) | 0 | (0.0) | 0 | (0.0) |
| Staphylococcal sepsis | 0 | (0.0) | 1 | (6.3) | 0 | (0.0) |
| Urinary tract infection | 1 | (6.3) | 0 | (0.0) | 0 | (0.0) |
| Injury, poisoning and procedural complications | 0 | (0.0) | 1 | (6.3) | 0 | (0.0) |
| Fall | 0 | (0.0) | 1 | (6.3) | 0 | (0.0) |
| Metabolism and nutrition disorders | 0 | (0.0) | 0 | (0.0) | 1 | (6.3) |
| Fluid overload | 0 | (0.0) | 0 | (0.0) | 1 | (6.3) |
| Musculoskeletal and connective tissue disorders | 1 | (6.3) | 0 | (0.0) | 0 | (0.0) |
| Pain in extremity | 1 | (6.3) | 0 | (0.0) | 0 | (0.0) |
| Nervous system disorders | 0 | (0.0) | 1 | (6.3) | 0 | (0.0) |
| Cerebral haemorrhage | 0 | (0.0) | 1 | (6.3) | 0 | (0.0) |
| Renal and urinary disorders | 1 | (6.3) | 0 | (0.0) | 0 | (0.0) |
| Nephrolithiasis | 1 | (6.3) | 0 | (0.0) | 0 | (0.0) |
| Respiratory, thoracic and mediastinal disorders | 1 | (6.3) | 0 | (0.0) | 0 | (0.0) |
| Dyspnoea | 1 | (6.3) | 0 | (0.0) | 0 | (0.0) |
| Vascular disorders | 2 | (12.5) | 0 | (0.0) | 0 | (0.0) |
| Hypotension | 2 | (12.5) | 0 | (0.0) | 0 | (0.0) |

NOTE:
Percentages are the proportions of subjects within that category.

Five subjects were reported as having SAEs during the study (Table 16). In the 100 mg/day treatment group, one subject had diarrhea of moderate severity and another subject had severe exacerbation of congestive heart failure (CHF). In the 200 mg/day treatment group, one subject had severe staphylococcal sepsis and a severe cerebral hemorrhage, another subject had a gastrointestinal hemorrhage of moderate severity, and a third subject had chest pain of moderate severity. No subject in the MCC 400 mg treatment group had an SAE. All SAEs were considered to be unrelated to study drug.

TABLE 16
Incidence of serious adverse events in the safety population of the 2A study

| | | | | | | | | | | |
|---|---|---|---|---|---|---|---|---|---|---|
| | | | | Total | | | | | | |
| | | | | Duration | | | | | | |
| Treatment | | | | of | | | Duration | | Relationship | |
| Subject | Age | | | Therapy | Adverse Event | Study | of AE | | to Study | |
| Number | (Yrs) | Sex | Race | (Days)^{a} | (Preferred Term) | Day^{b} | (Days) | Severity | Drug^{c} | Outcome |

MCC 100 mg/day

| | | | | | | | | | | |
|---|---|---|---|---|---|---|---|---|---|---|
| 314 | 34 | Male | Caucasian | 10 | Diarrhea | 33 | 3 | Moderate | Not Related | Recovered |
| | | | | | | | | | | Without |
| | | | | | | | | | | Sequelae |
| 400 | 52 | Male | Black | 10 | Cardiac Failure | 39 | 12 | Severe | Not Related | Recovered |
| | | | | | Congestive | | | | | With |
| | | | | | | | | | | Sequelae |

MCC 200 mg/day

| | | | | | | | | | | |
|---|---|---|---|---|---|---|---|---|---|---|
| 200 | 85 | Female | Caucasian | 10 | Staphylococcal | 10 | 7 | Severe | Not Related | Not Yet |
| | | | | | Sepsis | | | | | Recovered^{d} |
| | | | | | Cerebral | 10 | 7 | Severe | Not Related | Not Yet |
| | | | | | Haemorrhage | | | | | Recovered^{d} |
| 208 | 71 | Female | Caucasian | 10 | Gastrointestinal | 15 | 14 | Moderate | Not Related | Recovered |
| | | | | | Haemorrhage | | | | | Without |
| | | | | | | | | | | Sequelae |
| 304 | 59 | Female | Caucasian | 11 | Chest Pain | 23 | 6 | Moderate | Not Related | Recovered |
| | | | | | | | | | | Without |
| | | | | | | | | | | Sequelae |

^{a}Date of last dose of study medication minus date of first dose of study medication plus one.
^{b}Study day is calculated as follows: date of onset minus date of first date of study medication plus one.
^{c}Based on Investigator's assessment.
^{d}Subject died.

Pharmacokinetics
Plasma Concentration Data

In the phase 1B-MD study, after multiple dose oral administrations, plasma concentrations of MCC were mostly below the limit of quantification across the dose range

Detectable plasma concentrations were found only in 12 samples from 6 subjects.

Of the 12 detectable concentrations, only 2 were significantly above the LLOQ, while others barely exceeded the LLOQ of 5 ng/mL.

These two concentrations (11.1 and 48.0 ng/mL) were observed in Subject 021 on Day 1, Hour 1 and just prior to the tenth dose on Day 10, respectively.

It is to be noted that the 150 mg dose produced no detectable concentrations.

Due to low MCC plasma levels across the dose range, there were insufficient plasma data points above LLOQ for pharmacokinetic analysis.

In the phase 2A study, after multiple dose oral administrations, plasma concentrations of MCC were mostly below the limit of quantification but with a dose dependent increase in the number of samples, and number of subjects, with measurable plasma concentrations.

Detectable plasma concentrations were found in 2/15 (13.3%) subjects in the MCC 100 mg/day treatment group, 9/16 (56.3%) subjects in the MCC 200 mg/day treatment group, and 13/17 (76.5%) subjects in the MCC 400 mg/day treatment group.

Observable MCC concentrations ranged from 9.45 to 12.3 ng/mL in the MCC 100 mg/day treatment group, 5.12 to 93.7 ng/mL in the MCC 200 mg/day treatment group, and 5.32 to 84.9 ng/mL in the MCC 400 mg/day treatment group.

Of the detectable concentrations of MCC in all treatment groups, the majority (35/41; 85.4%) were under 21 ng/mL.

Concentrations of MCC over 50 ng/mL were observed in only 2 subjects, one each in the 200 mg/day and 400 mg/day dosing groups.

Urinary Excretion Data of MCC

Levels of MCC in the urine in the phase 1B-MD study were all below the limit of quantification (LLOQ=5 ng/mL).

Fecal Concentration Data of MCC

Table 17 shows fecal concentrations from the 1B-MD study, normalized to the 150 mg dose; fecal MCC averaged 916.0 μg/g (138.4-1768.9 μg/g).

TABLE 17
Fecal concentrations of MCC in the phase 1B-MD study,
normalized to a 150 mg dose.

| | | | [MCC] |
|---|---|---|---|
| | Dose | [MCC] | (normalized) |
| Subject | (mg) | (μg/g) | (μg/g) |
| Range | 150-450 | 415.1-5306.8 | 138.4-1768.9 |
| Mean: | | | 916.0 |
| SD: | | | 450.2 |

For the phase 2A study, in the MCC 100 mg/day treatment group (n=11 samples sufficient), fecal MCC averaged 255.6 μg/g (range: 81.9-558.3 μg/g) at the end of treatment. In the MCC 200 mg/day treatment group (n=9 samples sufficient), fecal MCC averaged 441.7 μg/g (range: 11.7-786.7 μg/g). In the MCC 400 mg/day treatment group (n=13 samples sufficient), fecal MCC averaged 1433.3 μg/g (range: 389.0-3974.8 μg/g).

TABLE 18
Fecal concentrations of MCC at the end of treatment in the phase
2A study.

| | | [MCC] | [MCC] |
|---|---|---|---|
| Dose | | range | average |
| (mg/day) | N | (μg/g) | (μg/g) |
| 100 | 11 | 81.9-558.3 | 255.6 |
| 200 | 9 | 11.7-786.7 | 441.7 |
| 400 | 13 | 389.0-3974.8 | 1433.3 |

Conclusions

In summary, the present studies show that MCC is well-tolerated after multiple oral doses up to 450 mg, achieves high levels at the site of action, and shows promising results in the treatment of C. difficile-associated diarrhea.

This study also found 1) there were no treatment-emergent adverse events felt to be possibly drug related in either study, 2) after multiple dose oral administrations, low MCC levels were detected in plasma, most of which fell below the limit of quantification. Consequential to low plasma concentrations, no intact MCC was detected in the collected urine of the 1B-MD study. 3) by contrast, fecal levels in both studies were extremely high, exceeding 10,000 times the MIC₉₀ (0.125 μg/mL) versus C. difficile, 4) among 45 subjects treated with a full course of therapy, only four subjects were considered failures prior to or at the end of 10 days of therapy, 2 subjects in the 50-mg q12 hr and 2 subjects in the 100-mg q12 hr dose groups. No failures (0/16) were noted in the 200-mg q12 hr dose, 5) recurrence was observed in only 2 subjects following successful treatment. Both recurred approximately one month after the end of therapy, 6) although not statistically significant, the median time-to-cessation of diarrhea, showed a trend which suggested that higher doses may be more efficacious. Time-to-cessation of diarrhea was determined to be 5.5 days for the 50-mg q 12 hr dose group, 3.5 days for the 100-mg q 12 hr dose group, and 3.0 days for the 200-mg q 12 hr dose group.

### Example 3

### MCC is Selectively Effective Against C. difficile In-Vivo, and does not Affect Major Members of the Anaerobic Fecal Flora: Key to a Lower Relapse Rate.

To test the hypothesis that MCC is selectively active in-vivo against C. difficile and could be relatively sparing of the normal anaerobic fecal flora, quantitative stool cultures were performed on serial stool samples obtained from patients entered into a Phase 2A dose ranging clinical trial of MCC (now designated MCC). Optimal antibiotic therapy of C. difficile diarrhea should eradicate the vegetative forms of the pathogen, yet spare major components of the normal flora presumed to be responsible for colonization resistance.

Methods

Patients (n=32) were randomized to receive 50, 100 or 200 mg twice daily of MCC for 10 days. No prior therapy was given to 24 patients; 8 receive 1 or 2 doses of standard therapy. As ecologic controls, 7 additional patients were treated with vancomycin 125 mg qid for 10 days. Fresh stool samples were cultured 10^{-2,4,6,8} for C. difficilevegetative and spore forms; fecal filtrates were tested for cytotoxin B by cell assay. At study entry and day 10, aerobic and anaerobic fecal flora cultures, diluted 10^{-3,5,7,9}, were examined for major floral shifts. Since Bacteroides group organisms are ubiquitously present and cultivable, this genera was selected as a indicator of the integrity of the microbial flora.

Detailed method shows as the following.
1) Single center study in Calgary Health Region catchment area, population ˜1 million
2) Randomized open label, dose ranging Phase 2A study comparing 50 mg, 100 mg or 200 mg Q 12 hourly of MCC for 10 days p. o. as therapy of CDAD.
3) Following completion of the trial recruitment, a separate ecology control group of patients who otherwise would be eligible for the trial were treated with Vancomycin 125 mg QID for 10 days as a treatment/ecologic control.
4) Mild to moderate CDAD: >3 but <12 diarrheal samples/24 hours at study entry, positive C. difficile toxin assay, fever <39 degrees C., WBC <30,000/mm3, no vomiting, no severe abdominal discomfort
5) Primary CDAD or first relapse episode only.
6) Treatment naïve if possible. The protocol allowed up to 3 prior doses of standard therapy, but for this evaluation, a maximum of 2 doses of standard therapy was allowed. In this study population, 24 patients were treatment naive
7) No concomitant parenteral antibiotic therapy for any condition.
8) Serial stool samples: in addition to the original diagnostic sample, a repeat collection of stool >5 grams (10-30 grams usually) was obtained at study entry, at day 4, 7,10, 14, 21, 28 and 42 days after study entry
9) For this report, results of day 0 and day 10 stools are compared for changes in C. difficile counts and in counts of major genera of the normal colonic flora.
10) C. difficile quantitative counts and fecal filtrate concentrations of C. difficile cytotoxin B by HeLa cell assay were determined with freshly passed samples as refrigeration is deleterious to determination of quantitative counts of C. difficile.
11) Since Bacteroides group organisms are considered to be uniformly present in subjects and in high counts, and is likely one of the major components of the normal flora conferring ‘colonization resistance’, this group was used as an index of suppression of the anaerobic fecal flora. For patients who failed to show return of the Bacteroides group species at 10 days, subsequent samples were processed to document time of return of this group. If samples were not immediately processed, aliquots were frozen at −80 degrees C. with 15% glycerol/Brain Heart Infusion Broth for subsequent processing.
12) Media and methods for anaerobic flora cultures are based on the Wadsworth-KTL Anaerobic Manual, 6th ed, 2002. C. difficile counts were determined by dilution of the sample 10^{-2,4,6,8}/gram stool wet weight on CCFA agar. Spore counts were determined by treating an aliquot of stool with an equal volume of 100% ethyl alcohol ×1 hour, centrifuged, washed twice and resuspended for quantitative counts.
13) Normal flora cultures were quantified by dilution 10^{-3,5,7,9} using MacConkey, BAP, m-Enterococcus agar, Lab M anaerobic blood agar, BAP, BBE, KVLB, PEA agars incubated for 48 hours before initial inspection, and further incubated for up to 7 days.
14) For vancomycin ecologic controls, vancomycin fecal filtrate concentrations were determined in triplicate by bioassay using a C. perfringens as the indicator organism.
15)Differences in microbial counts were determined after log₁₀ transformation using wilcoxon matched.
   Results

At study entry, mean log₁₀ CFU±SD vegetative counts of C. difficile (all MCC patients) were 6.8±3.6, range 2-10.95; at day 10, with the exception of one patient receiving 50 mg, all other patients had C. difficile quantitative counts reduced <2 log₁₀/gm feces. Vancomycin was similarly effective. At study entry, Bacteroides group counts were <3, 3-8, & 8.5-10 log₁₀ CFU/gm in 1/3 each of patients, with normal counts being >11. Shifts in the Bacteroides group are shown table 19.

TABLE 19
Mean ± SD of log₁₀ CFU of Bacteroides
group counts/gm feces wet weight

| | MCC, 50 mg | MCC, 100 mg | MCC, 200 mg | Vancomycin |
|---|---|---|---|---|
| | (n = 10) | (n = 8) | (n = 11) | (n = 7) |
| Day 0 | 6.64 ± 2.82 | 6.64 ± 2.82 | 7.04 ± 2.87 | 7.39 ± 2.67 |
| Day 10 | 8.23 ± 2.60 | 6.30 ± 2.53 | 7.34 ± 3.06 | 3.62** ± 1.90 |
| p* | 0.11 | 0.44 | 0.56 | 0.03 |

*wilcoxon matched pairs signed-ranks test, 2 tailed;
**counts <3 log₁₀ = 2.90

The following figures further illustrate the results from the study.

Conclusions

Based on quantitative Bacteroides group counts, patients with C. difficile diarrhea have variably impaired normal flora at study entry, with approximately 1/3 in the 3 log₁₀ CFU/gm range, 1/3 in counts of 4-7 log₁₀ CFU, and the remainder with higher counts (none in the normal range of 11-12 log₁₀ CFU). All dosages of MCC appeared to reduce counts of C. difficile, as did vancomycin. A dose dependent reduction in Bacteroides counts with increasing dosages of MCC was not observed. Vancomycin severely impairs Bacteroides counts during therapy and although most patients recover their counts, a minority have prolonged absence.

Based on these data and clinical outcomes showing a high response rate accompanied by a low relapse rate, it would appear that the 200 mg dose of MCC would be an appropriate dosage to undergo further clinical investigation.

The invention is not limited by the embodiments described above which are presented as examples only but can be modified in various ways within the scope of protection defined by the appended patent claims

We claim:

1. A method of treating a disease or disorder caused by the presence of a bacterium comprising administering to a patient in need an effective amount of a mixture, where the mixture comprises an effective amount of tiacumicin B and an additional macrocycle selected from the group consisting of: 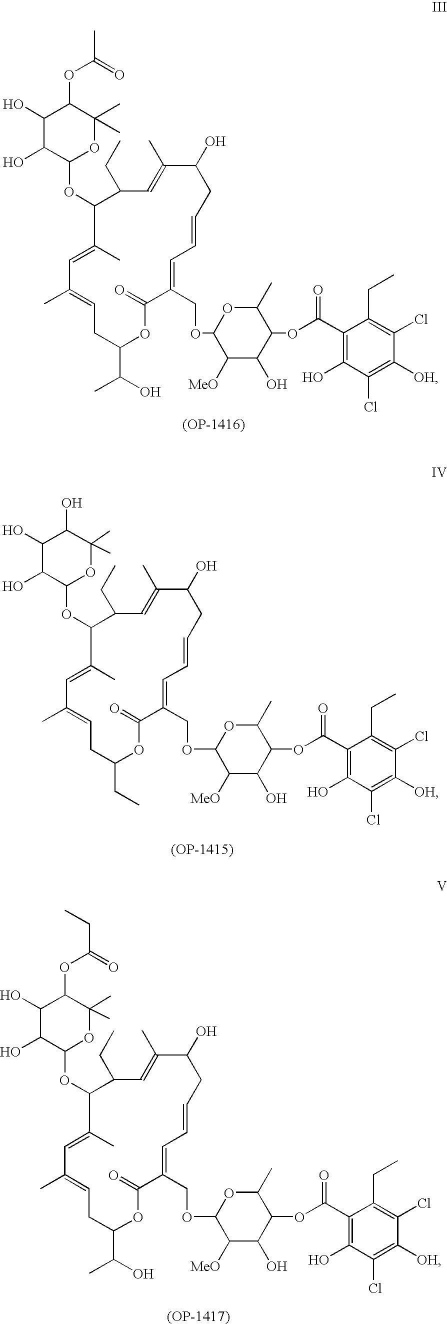 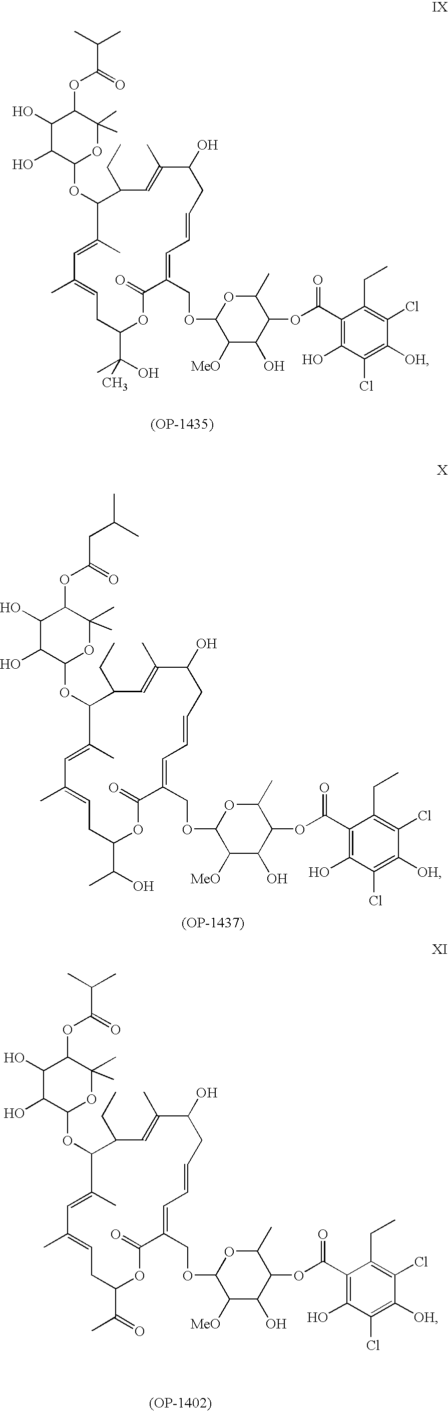 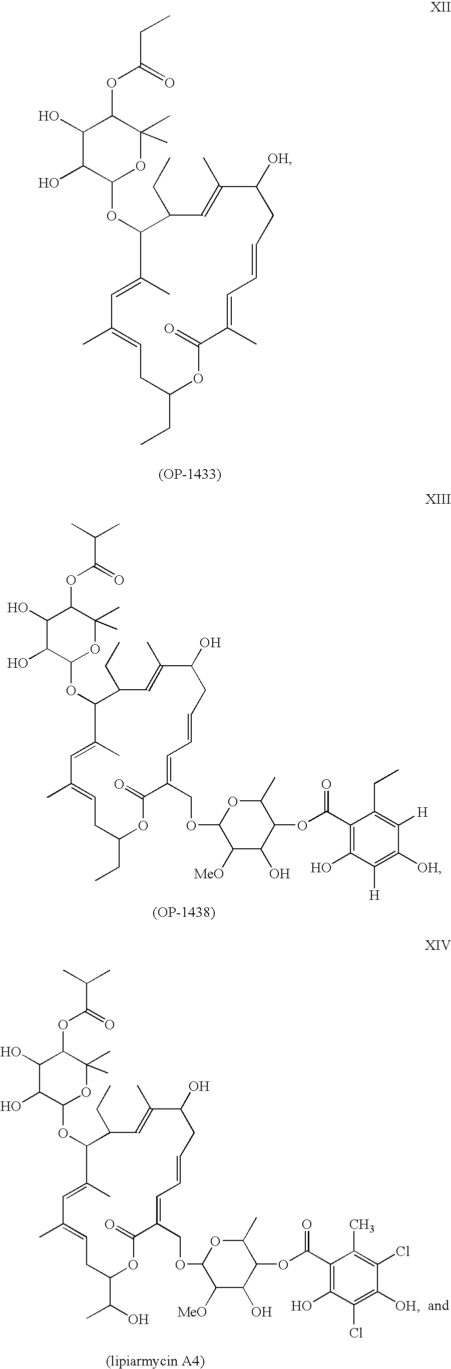
combinations thereof, wherein when the compound of formula XIV is present, the mixture comprises about 0.1% to about 5% of the compound of formula XIV by weight.

2. The method of claim 1 wherein the mixture comprises at least 90% of tiacumicin B by weight.

3. The method of claim 1 wherein the mixture comprises at least 95% of tiacumicin B by weight.

4. The method of claim 1 wherein the mixture comprises at least 98% of tiacumicin B by weight.

5. The method of claim 1 wherein the mixture comprises at least 1% by weight of the additional macrocycles in total.

6. The method of claim 1 wherein the mixture comprises from about 2% to about 5% of the additional macrocycles in total.

7. The method of claim 1 wherein the mixture exhibits an HPLC profile substantially depicted at FIG. 5.

8. The method of claim 1 wherein the mixture comprises about 0.3 to about 5% of the compound of formula XIV by weight.

9. The method of claim 1 wherein the mixture comprises about 0.3 to about 3% of the compound of formula XIV by weight.

10. The method of claim 1 wherein the mixture comprises about 0.3 to about 1.5% of the compound of formula XIV by weight.

11. The method of claim 1 wherein the mixture comprises about 1% of the compound of formula XIV by weight.

12. The method of claim 1 wherein the mixture further comprises at least one of the following compounds:

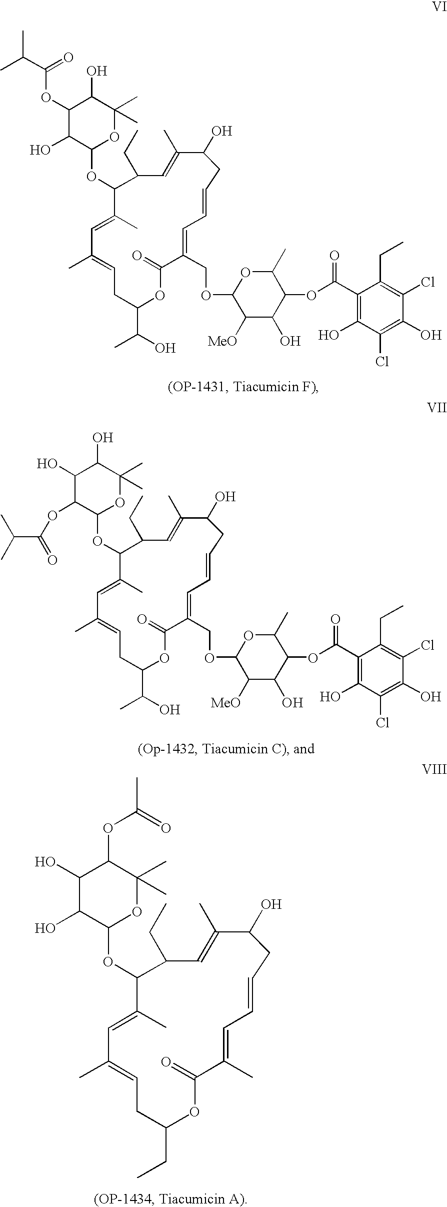

13. The method of claim 1 wherein the bacterium is selected from the group consisting of Clostridium species, Staphylococcus species, Enterococcus species and combinations thereof.

14. The method of claim 1 wherein the bacterium is selected from C. difficile, C. perfringens, S. aureus, and combinations thereof.

15. The method of claim 1 wherein the bacterium is C. difficile.

16. The method of claim 1 wherein the mixture does not substantially affect major members of the anaerobic gastrointestinal flora in the patient.

17. The method of claim 1 wherein the relapse rate of the disorder or disease is substantially reduced.

18. The method of claim 1 wherein the disease is at least one of diarrhea and colitis.

19. The method of claim 1 wherein the disease is infectious diarrhea.

20. The method of claim 18 wherein the disease is Clostridium difficile-associated diarrhea.

21. The method of claim 1 wherein the mixture is prepared by a process comprising:
culturing a microorganism in a nutrient medium to accumulate the mixture in the nutrient medium; and
isolating the mixture from the nutrient medium;
wherein the nutrient medium comprises an adsorbent to adsorb the mixture.

22. The method of claim 21 wherein the nutrient medium comprises about 0.5 to about 15% of the adsorbent by weight.

23. The method of claim 21 wherein the absorbent is an adsorbent resin.

24. The method of claim 23 wherein the adsorbent resin is selected from the group consisting of Amberlite® XAD16, XAD16HP, XAD2, XAD7HP,XAD1 180, XAD1600, IRC50, and Duolite® XAD761.

25. The method of claim 21 wherein the microorganism is Dactylosporangium aurantiacum subspecies hamdenensis.

26. The method of claim 1 wherein the disease is associated with the use of antibiotics or cancer chemotherapies or antiviral therapy.

27. The method of claim 1 wherein the Staphylococcus species is methicillin-resistant Staphylococcus species.

28. The method of claim 1 wherein the Staphylococcus species is methicillin-resistant Staphylococcus aureus.

29. The method of claim 1 wherein the Enterococcus species is vancomycin-resistant Enterococcus.

30. The method of claim 1 wherein the mixture is administered in an amount of about 50 mg to about 1000 mg one to three times daily within three to fifteen days.

31. The method of claim 1 wherein the mixture is administered in an amount of about 100 mg to about 400 mg once or twice daily.

32. The method of claim 1 wherein the mixture is administered in an amount of about 200 mg once daily.

33. The method of claim 1 wherein the mixture is administered in an amount of about 200 mg twice daily.

34. The method of claim 1 wherein the mixture is administered in a manner so that the plasma concentration of the mixture in the patient is below 5 ng/mL.

35. The method of claim 1 wherein the mixture is administered in a manner so that the concentration of the mixture in the urine of the patient is below 5 ng/mL.

36. A pharmaceutical mixture comprising tiacumicin B and an additional macrocycle selected from the group consisting of: 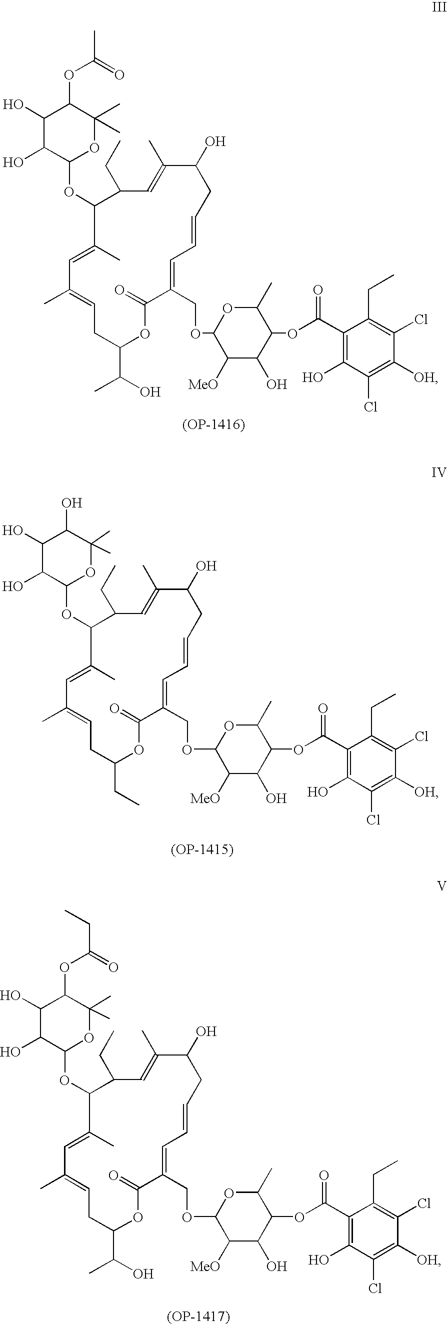 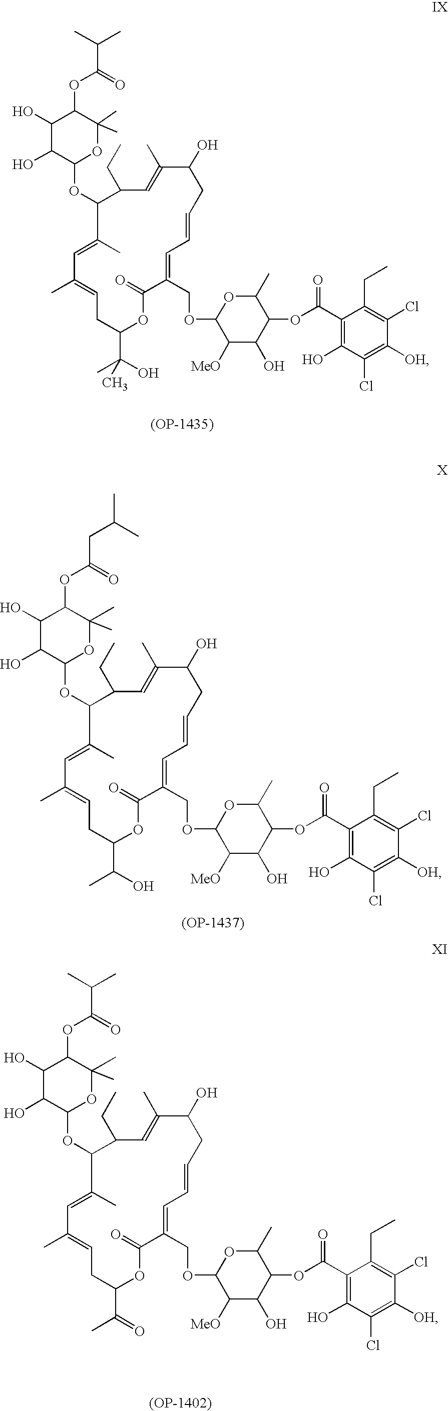 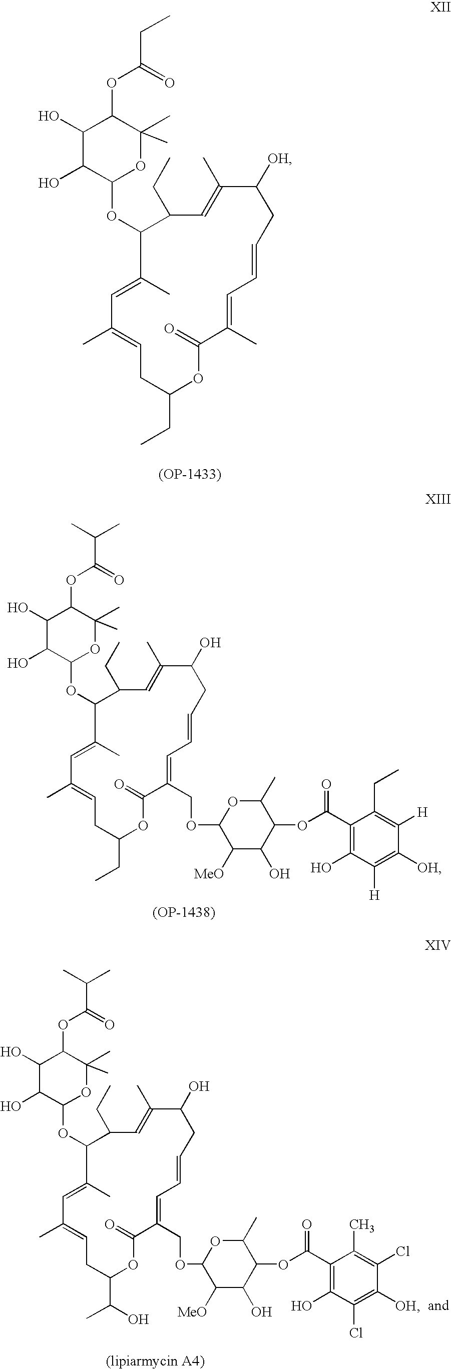
combinations thereof, wherein when compound of formula XIV is present, the mixture comprises about 0.1 to about 5% compound of formula XIV.

37. The mixture of claim 36 comprising at least 90% of tiacumicin B by weight.

38. The mixture of claim 36 comprising at least 95% of tiacumicin B by weight.

39. The mixture of claim 36 comprising at least 1% of the additional macrocycles by weight in total.

40. The mixture of claim 36 comprising from about 2% to about 5% of the additional macrocycles by weight in total.

41. The mixture of claim 36 wherein the mixture exhibits a HPLC profile substantially depicted at FIG. 5.

42. The mixture of claim 36 wherein the mixture comprises about 0.3% to about 5% of the compound of formula XIV by weight.

43. The mixture of claim 36 wherein the mixture comprises about 0.3% to about 3% of the compound of formula XIV by weight.

44. The mixture of claim 36 wherein the mixture comprises about 0.3% to about 1.5% of the compound of formula XIV by weight.

45. The mixture of claim 36 wherein the mixture comprises about 1% of the compound of formula XIV by weight.

46. The mixture of claim 36 further comprising at least one of the following compounds: [chemical structure]
